(19) United States
(12) Patent Application Publication
(10) Pub. No.: US 2005/0022426 A1
(43) Pub. Date: Feb. 3, 2005

(54) Shoe construction

(75) Inventors: Douglas E. Clark, Durham, NH (US); Stephen D. Opie, Arlington, MA (US); Bert Appleton Spiller, Dover, NH (US); David E. Miller, Dayton, ME (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: The Timberland Company, Stratham, NH (US)

(21) Appl. No.: 10/932,947

(22) Filed: Sep. 2, 2004

Related U.S. Application Data
(63) Continuation of application No. 10/025,211, filed on Dec. 19, 2001.
(60) Provisional application No. 60/258,184, filed on Dec. 22, 2000.
(60) Provisional application No. 60/304,250, filed on Jul. 10, 2001.

(51) Int. Cl. A43B 13/12; A43B 7/16; A43B 23/00; A61F 5/14

(52) U.S. Cl. 36/30 R; 36/107; 36/173; 36/174

(57) ABSTRACT

A shoe has a bottom including an expansion mechanism for expanding the bottom laterally underlying the metatarsals. An upper is carried by the bottom. A chassis is carried by the bottom for underlying the foot. In one embodiment, the expansion mechanism of the bottom includes a base portion and an expansion portion, the base portion having a plurality of slots extending generally longitudinally in the mid-foot region defining the expansion portion, the expansion portion formed of a material allowing lateral expansion of the bottom. In one embodiment, the upper has an expanding and contracting mechanism for expanding and contracting the upper therein stabilizing holding the foot as the foot moves. In one embodiment, the expanding and contracting mechanism is an elastic omega seam interposed between two pieces of leather at a seam wherein the omega opens and closes. In one embodiment, the chassis has at least three tailored zones, a heel zone underlying the heel of the foot, a metatarsal zone underlying the metatarsals, and a toe zone underlying the toes of the foot. In one embodiment, the chassis has an additional tailored zone, an arch zone underlying the arch of the foot.

### CROSS-REFERENCE TO RELATED APPLICATIONS

The present application is a continuation of U.S. patent application Ser. No. 10/025,211, filed Dec. 19, 2001, which application claims the benefit of the filing dates of U.S. Provisional Application No. 60/304,250, filed on Jul. 10, 2001 and U.S. Provisional Application No. 60/258,184, filed on Dec. 22, 2000, the disclosures of which are hereby incorporated herein by reference.

### BACKGROUND OF THE INVENTION

People have been wearing articles on their feet for protection for thousands of years. In the twentieth century these articles, typically referred to as shoes, have included three basic components: a bottom which contacts the ground and creates a barrier from the ground, an inner sole which contacts the foot, and an upper portion which primarily retains the foot within the shoe. The three components can vary and be integrated; for example, a flat sandal wherein the bottom and the sole are formed together as one piece of homogenous material and the upper consists of a few straps to retain the foot against the sole. In addition, shoes can have other special features such as added support or protection. The protection feature can be, for example, a layer of hardened material over the toe for protection in a work environment.

Shoes have been classified in numerous different ways in the latter half of the twentieth century. One such classification system consists of the three categories of conventional, Euro Comfort, and fashion. The first category, that of conventional shoes, typically has stiff leather components which result in a stiff shoe that retains its shape. The second category, that of Euro Comfort shoe, includes shoes where the leather is soft and generally has a wide last, track of a foot or form. In addition, the Euro Comfort shoe typically has foam as part of the insole under the foot. The Euro Comfort type shoes typically do not hold their shape as in conventional shoes. The third category is that of fashion shoe wherein the desired feature is the look of the shoe with little concern related to comfort or the health of the foot.

### SUMMARY OF THE INVENTION

One embodiment of the present invention provides footwear including a bottom; an upper carried by the bottom to define a foot receiving cavity; and a chassis inserted in the foot receiving cavity in overlying relationship to the bottom. The bottom has an upper layer and a lower layer, the upper layer having an opening defining a void space between the lower layer and the chassis, and at least one insert assembled in the void space. The upper layer is formed from a first material and the insert is formed from a second material different from the first material. Preferably, the opening in the upper layer is in a position underlying the forefoot of a foot when the foot is in the foot receiving cavity.

In one variant of this embodiment, the insert may include a first inlay and a second inlay, the first inlay being formed from one material and the second inlay being formed from another material different from the one material. Preferably, the first inlay is in a position underlying the toes of a foot when the foot is in the foot receiving cavity, and the second inlay is in a position underlying the metatarsals of the foot when the foot is in the foot receiving cavity.

The one material forming the first inlay may have a first degree of hardness and the another material forming the second inlay may have a degree of hardness less than the first degree of hardness. Preferably, the one material is a first polyurethane and the another material is a polyurethane different from the first polyurethane.

In another variant of this embodiment, the insert may be formed from a displacement material and the lower layer may include a bottom surface and a plurality or through ports extending from the upper layer to the bottom surface. The displacement material preferably has a degree of deformability such that a portion of the displacement material is displaced into at least some of the through ports as a load is applied to the displacement material in a direction toward the lower layer.

Another embodiment of the present invention provides footwear including a bottom; an upper carried by the bottom to define a foot receiving cavity; and a chassis inserted in the foot receiving cavity in overlying relationship to the bottom. The bottom has an upper layer, a lower layer, a void space defined between the upper layer and the lower layer, and a displacement material positioned in the void space. Preferably, the void space is in a position underlying the forefoot of a foot when the foot is in the foot receiving cavity.

When no downward load is applied to the upper material of the bottom, the void space may have an initial volume and the displacement material may have a volume less than the initial volume. In preferred arrangements, a bottom surface of the upper layer includes a generally concave recess, and the displacement material is placed below the generally concave recess.

### BRIEF DESCRIPTION OF THE DRAWINGS

The foregoing and other objects, features, and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a top medial side perspective view of a right human foot;

FIG. 2 is a bottom view of a right human foot;

FIG. 3 is a top medial side perspective view of a left shoe according to the invention;

FIG. 4A is a view similar to FIG. 3 with the top portion broken away and rotated so the inner portion is seen. The chassis of the toe portion is exploded out;

FIG. 4B is a cross-sectional view of the toe portion of FIG. 4A;

FIG. 5 is a bottom view of a bottom of a shoe according to the invention;

FIG. 6A is a sectional view of the bottom of the shoe taken along the line 6A-6A of FIG. 5;

FIG. 6B is a sectional view of the bottom of the shoe taken along the line 6B-6B of FIG. 5;

FIG. 6B1 is an enlarged view of one of the lips shown in FIG. 6B;

FIG. 6C is a sectional view of the bottom of the shoe taken along the line 6C-6C of FIG. 5;

FIG. 6D is a sectional view of the bottom of the shoe taken along the line 6D-6D of FIG. 5;

FIG. 6E is a sectional view of the bottom of the shoe taken along the line 6E-6E of FIG. 5;

FIG. 7A is a sectional view of the bottom of the shoe taken along the line 7A-7A of FIG. 5;

FIG. 7B is a sectional view of the bottom of the shoe taken along the line 7B-7B of FIG. 5;

FIG. 8A is a top view of a chassis of a shoe;

FIG. 8B is a bottom view of a chassis of a shoe;

FIG. 9A is a sectional view of the chassis of the shoe taken along the line 9A-9A of FIG. 8B;

FIG. 9B is a sectional view of the chassis of the shoe taken along the line 9B-9B of FIG. 8B;

FIG. 9C is a sectional view of the chassis of the shoe taken along the line 9C-9C of FIG. 8B;

FIG. 9D is a sectional view of the chassis of the shoe taken along the line 9D-9D of FIG. 8B;

FIG. 9E is a sectional view of the chassis of the shoe taken along the line 9E-9E of FIG. 8B;

FIG. 9F is a sectional view of the chassis of the shoe taken along the line 9F-9F of FIG. 8B;

FIG. 9G is a sectional view of the chassis of the shoe taken along the line 9G-9G of FIG. 8B;

FIG. 10A is a lateral side view of the chassis;

FIG. 10B is a medial side view of the chassis;

FIG. 11A is a sectional view showing an elastic omega seam;

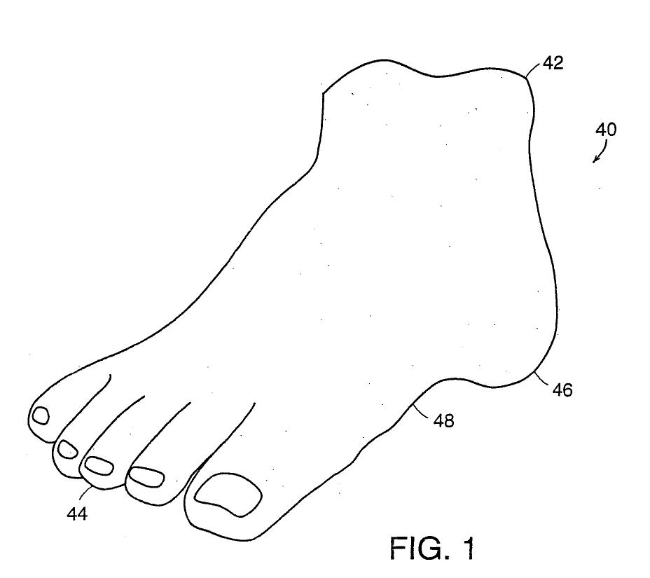

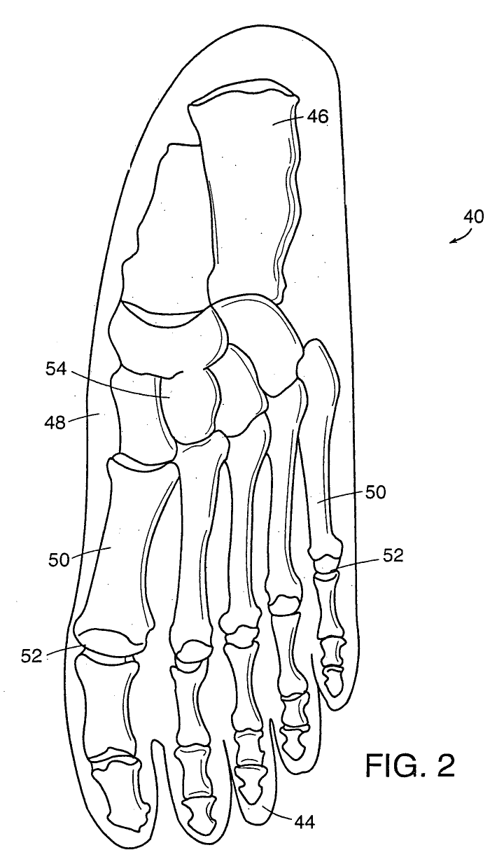

FIG. 1B is a sectional view of the elastic omega seam in a stretched position;

FIG. 12A is a sectional view showing a piece of a goring connecting the side to the upper portion of the upper;

FIG. 12B is a sectional view showing the goring of FIG. 12A in a stretched position;

FIG. 13A is a side view of the waterproof, breathable membrane on the last prior to the taping;

FIG. 13B is a side view of a waterproof, breathable membrane on a last;

FIG. 13C is a bottom view of the waterproof, breathable membrane with a gasket on the last;

FIG. 13D is a bottom side perspective view of the waterproof, breathable membrane on the last;

FIG. 13E is a front lateral perspective view of an alternative construction of a waterproof, breathable membrane;

FIG. 14A is an alternative construction of a waterproof, breathable membrane;

FIG. 14B is the waterproof, breathable membrane of FIG. 14A including the gasket;

FIG. 14C is an alternative construction of a waterproof, breathable membrane;

FIG. 14D is the waterproof, breathable membrane of FIG. 14C with a full gasket;

FIG. 14E is a bottom view of an alternative construction of a waterproof, breathable membrane with a string lost construction;

FIG. 14F is a sectional view taken along the lines 14F-14F of FIG. 14E;

FIG. 15A is a top view of an alternative chassis;

FIGS. 15B-15F are schematic sectional views of the chassis of FIG. 15A for five alternative embodiments;

FIG. 16A is a top view of an alternative embodiment of a chassis of a shoe;

FIG. 16B is a bottom view of the alternative embodiment of a chassis of a shoe of FIG. 16A;

FIG. 17A is a sectional view of the chassis of the shoe taken along the line 17A-17A of FIG. 16B;

FIG. 17B is a sectional view of the chassis of the shoe taken along the line 17B-17B of FIG. 16B;

FIG. 18A is a sectional view of the chassis of the shoe taken along the line 18A-18A of FIG. 16B;

FIG. 18B is a sectional view of the chassis of the shoe taken along the line 18B-18B of FIG. 16B;

FIG. 18C is a sectional view of the chassis of the shoe taken along the line 18C-18C of FIG. 16B;

FIG. 18D is a sectional view of the chassis of the shoe taken along the line 18D-18D of FIG. 16B;

FIG. 18E is a sectional view of the chassis of the shoe taken along the line 18E-18E of FIG. 16B;

FIG. 18F is a sectional view of the chassis of the shoe taken along the line 18F-18F of FIG. 16B;

FIG. 18G is a sectional view of the chassis of the shoe taken along the line 18G-18G of FIG. 16B;

FIG. 19A is a lateral side view of the chassis;

FIG. 19B is a medial side view of the chassis;

FIG. 19C is a side sectional view of a shoe with the chassis linked to the upper;

FIG. 19D is a side view of an alternative shoe with an alternative linking mechanism between the chassis and the upper;

FIG. 19E is a rear view of the shoe of FIG. 19D;

FIG. 19F is an enlarged view of a linking mechanism;

FIG. 20 is a front side view of an alternative shoe;

FIG. 21A is a bottom view of the shoe of FIG. 20;

FIG. 21B is a top view of the bottom of the shoe;

FIG. 22A is a sectional view of the bottom of the shoe taken along the line 22A-22A of FIGS. 21A and 21B;

FIG. 22B is a sectional view of the bottom of the shoe taken along the line 22B-22B of FIGS. 21A and 211B;

FIG. 22B1 is a sectional view of the shoe including the upper and the chassis taken along the line 22B-22B;

FIG. 22C is a sectional view of the bottom of the shoe taken along the line 22C-22C of FIGS. 21A and 211B;

FIG. 22D is a sectional view of the bottom of the shoe taken along the line 22D-22D of FIGS. 21A and 211B;

FIG. 22D1 is an enlarged view of one of the lips shown in FIG. 22D;

FIG. 22E is a sectional view of the bottom of the shoe taken along the line 22E-22E of FIGS. 21A and 211B;

FIG. 23A is a medial side view of the bottom of FIGS. 21A and 21B;

FIG. 23B is a lateral side view of the bottom;

FIG. 24A is a bottom view of an alternative embodiment of a bottom of a shoe;

FIG. 24B is a top view of the bottom of the shoe of FIG. 24A;

FIG. 25A is a sectional view of the bottom of the shoe taken along the line 25A-25A of FIG. 24A;

FIG. 25B is a sectional view of the bottom of the shoe taken along the line 25B-25B of FIG. 24A;

FIG. 25C is a sectional view of the bottom of the shoe taken along the line 25C-25C of FIG. 24;

FIG. 26 is a front sectional view of a shoe with an alternative bottom construction;

FIG. 27A is a front sectional view of a shoe with another alternative bottom construction; and

FIG. 27B is a top view of a front portion of the bottom of the shoe of FIG. 27A.

### DETAILED DESCRIPTION

A foot 40 is shown in FIGS. 1 and 2. The foot 40 has several key and distinct regions going from an ankle 42 to the tip of a toe 44. These regions of the foot 40 include a heel 46, an arch 48 on the medial side of the foot 40, and the toes 44. In addition, as best seen, FIG. 2, the foot 40 has five metatarsals 50, the portion of the foot 40, more particularly the bone of the foot 40, that extend rearward from each of the respective metatarsophalangeal (MP) joints 52 to the anterior portion of the tarsus 54 formed by the distal aspect of the cuneiforms and cuboid.

The foot 40 is far from homogeneous and the regions have different characteristics. The heel 46 is predominantly a boney area, while the arch 48 is predominantly connective tissue. The toes 44 in contrast are more brittle and need to have space to be able to spread apart and move to aid balance, adapt to varying terrain, and manage ground reaction forces and the resultant pressure.

In conventional shoes, loading of the weight of the body through the foot 40 is typically at the heel 46 and the first and the fifth MP joints 52. The heel 46 has the greatest loading. The first MP joint 52 has the second greatest concentration of loading.

It is recognized that the shape of the foot is dynamic and the foot's shape changes as the foot loads and unloads. The shape of the foot when an average person is standing is different than that of the same foot when a person is sitting and applying no weight to the foot. The foot 40 of an average human adult in the metatarsal region can expand up to 5 millimeters from a resting position to a standing position. This expansion of 3 to 5 millimeter relates to a typical half (½) a shoe size in width.

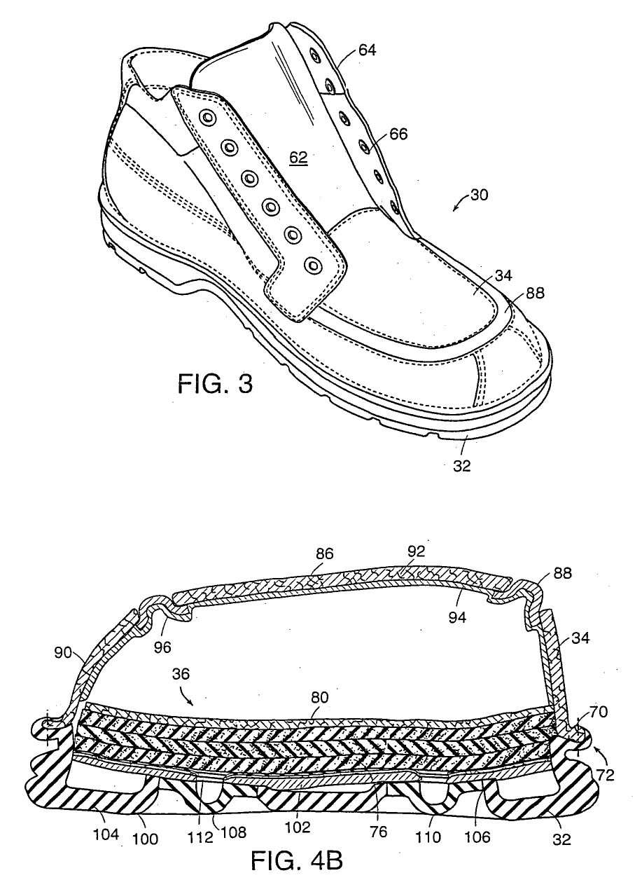

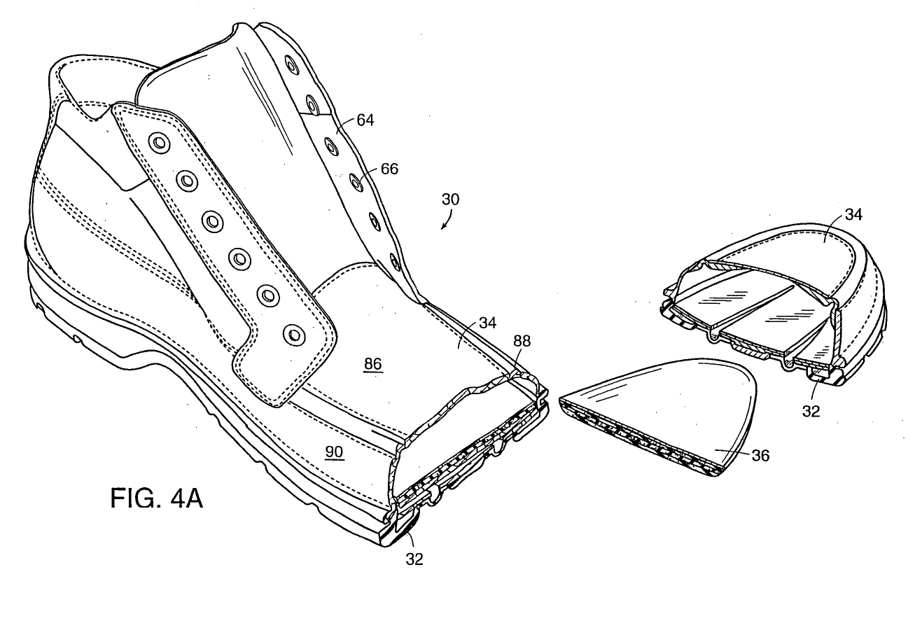

FIG. 3 is a perspective view of a shoe 30 according to the invention. The shoe 30 is shown from a top medial side view. The shoe 30 has three main components, a bottom 32, an upper 34, and a chassis 36 as seen in FIG. 4A. The upper 34 consists predominantly of leather and in the embodiment shown has a tongue 62 and a pair of eyelet tabs 64 each with a plurality of eyelets 66 for receiving a shoelace. The upper 34 is secured to the bottom 32. In the embodiment shown, the upper 34 is secured to the bottom 32 with at least one line of stitching 70. The stitching 70 extends through the upper 34 and a portion of the bottom 32 and is referred to as the welt 72 as seen in FIG. 4B.

FIG. 4A is a perspective view of the shoe 30 of FIG. 3 with the toe portion of the shoe 30 broken away and rotated in order to see the interior. The chassis 36 is seen carried on top of the bottom 32 and encircled by the upper 34.

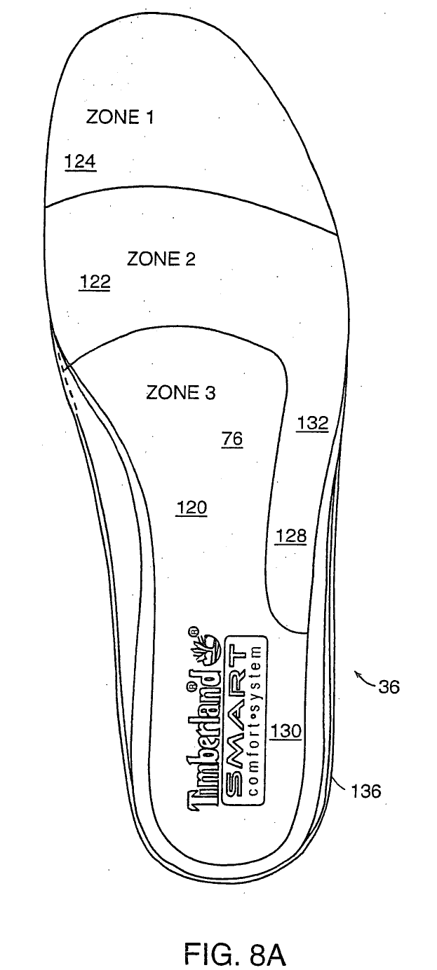

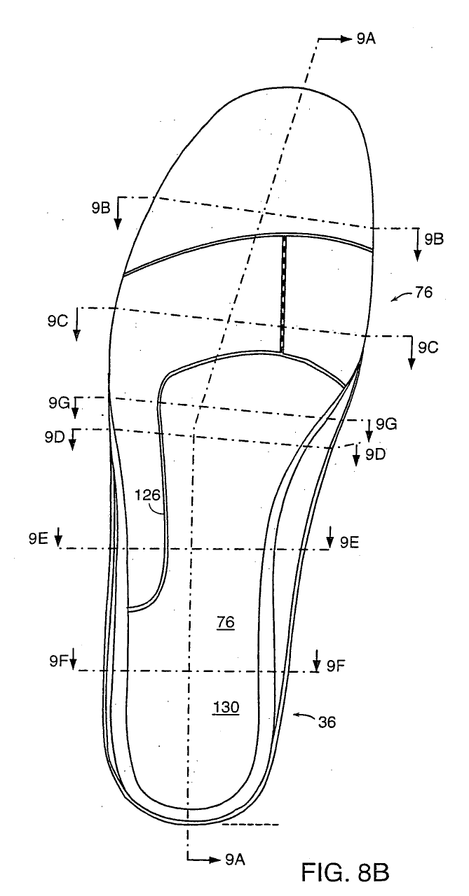

As explained in greater detail below, the chassis 36 in this embodiment, as seen in FIGS. 4A and 4B, has a series of layers including at least a base layer 76 and an upper layer 80. The chassis 36 is described below with respect to FIGS. 8A-10B.

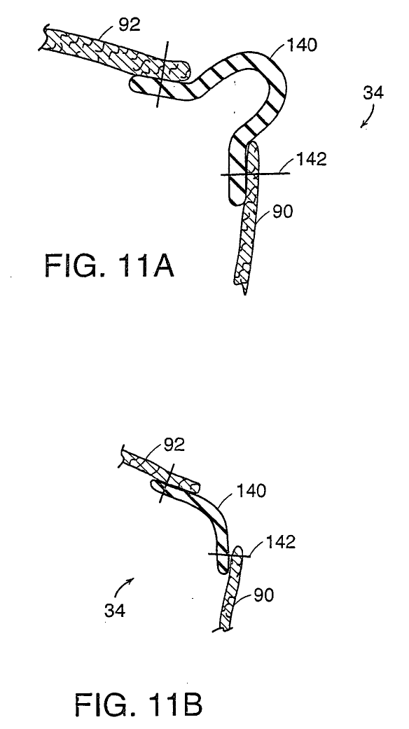

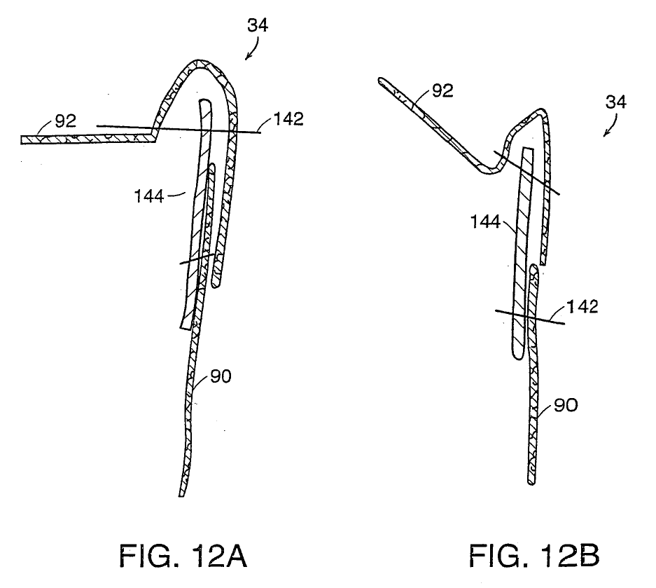

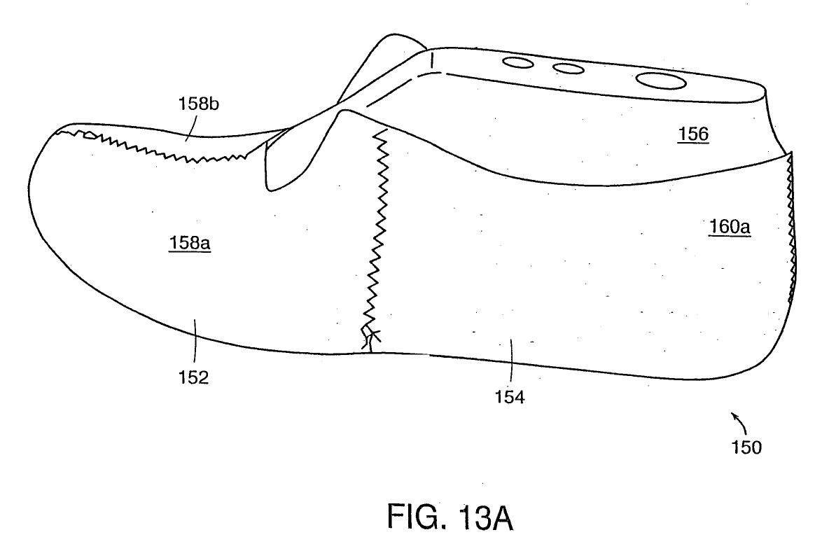

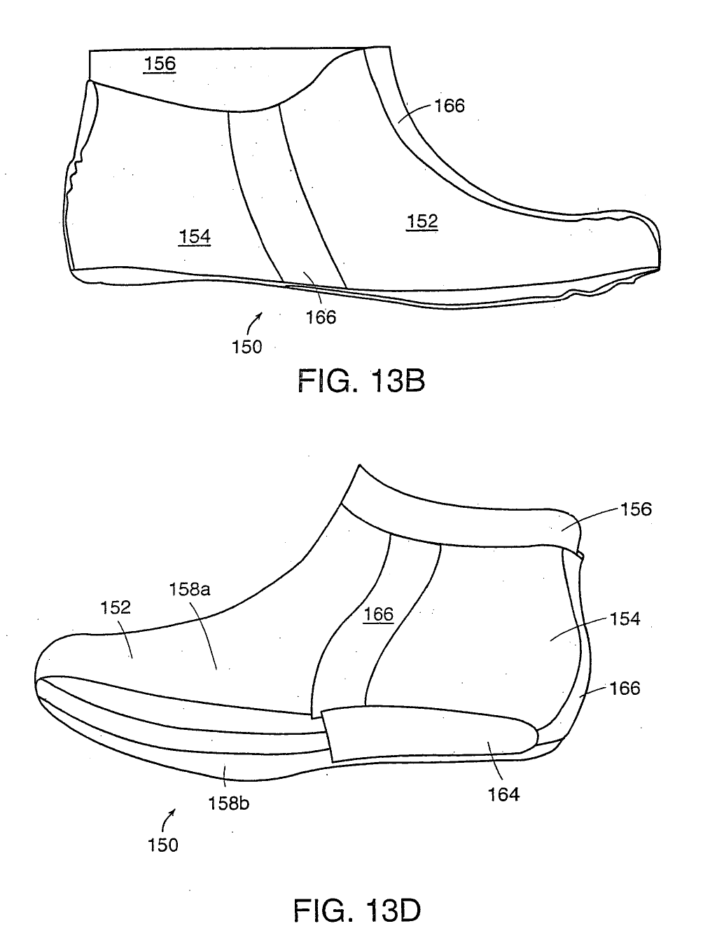

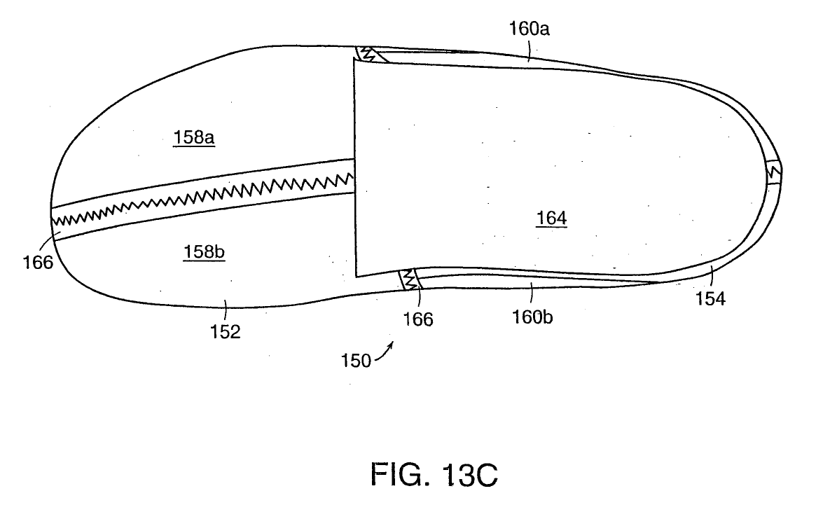

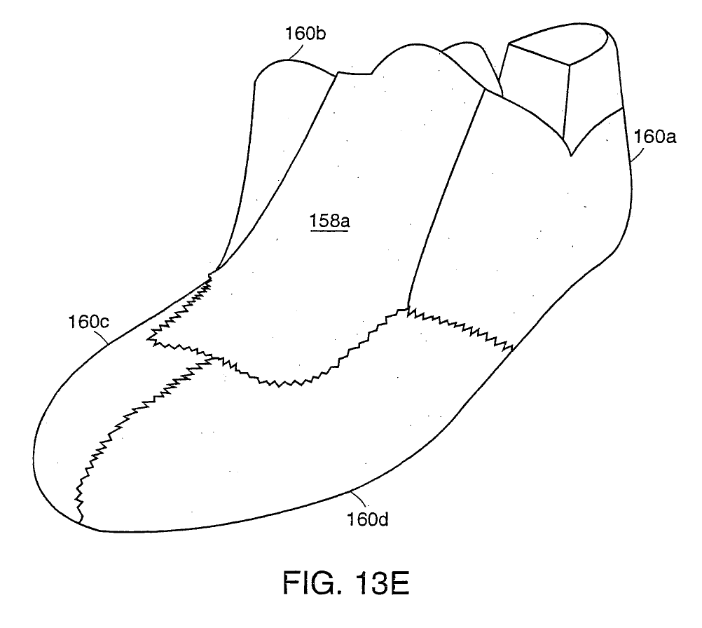

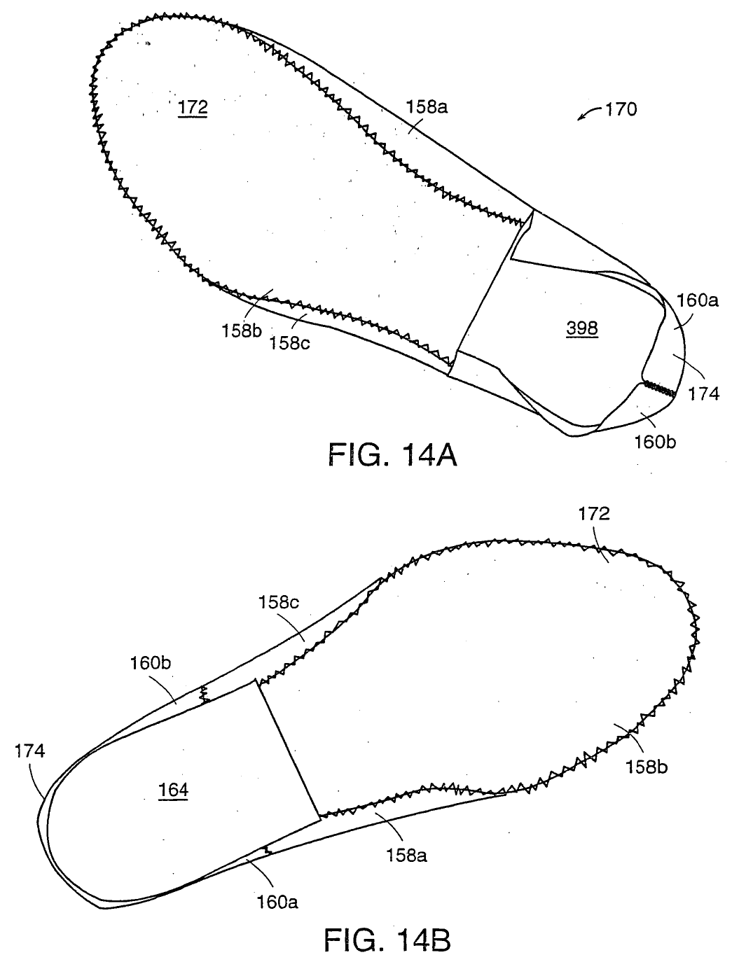

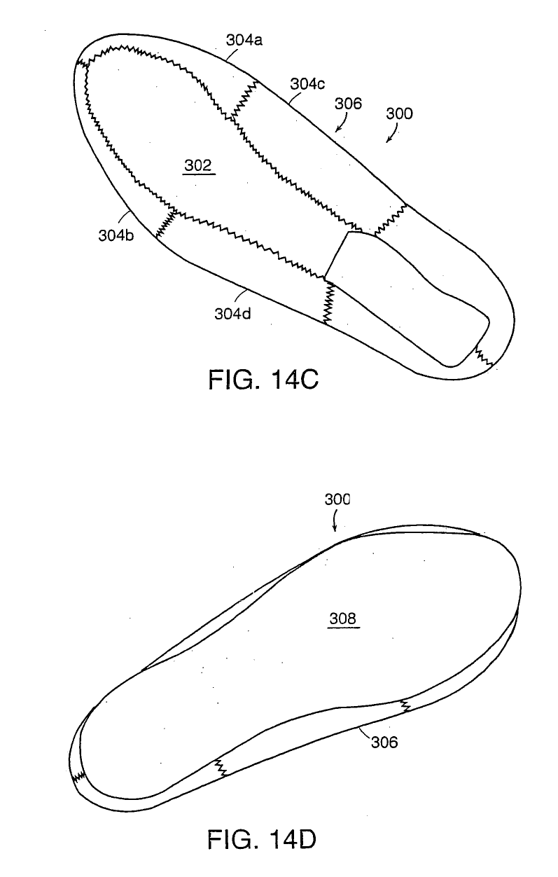

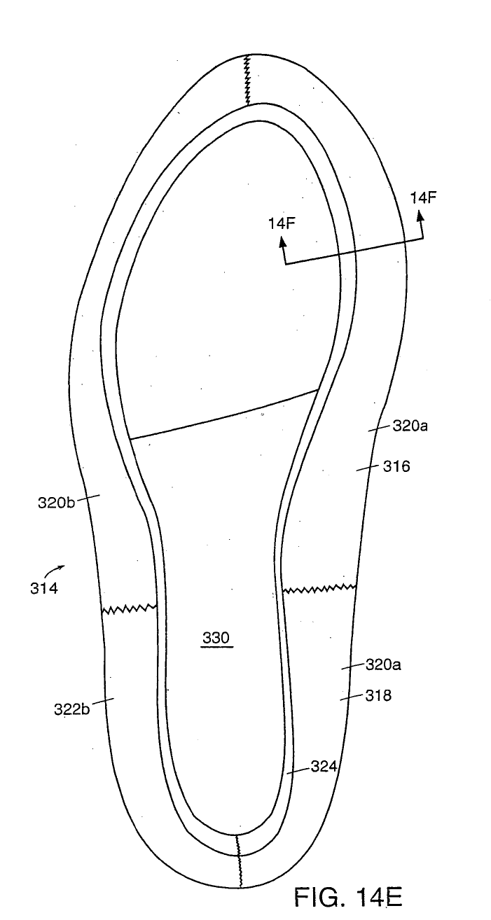

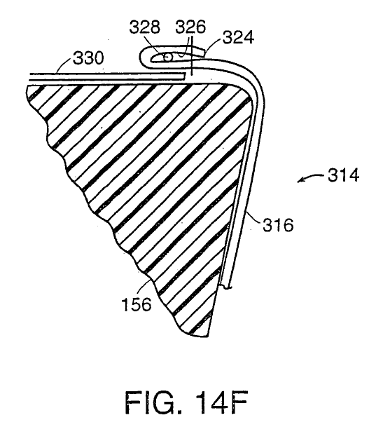

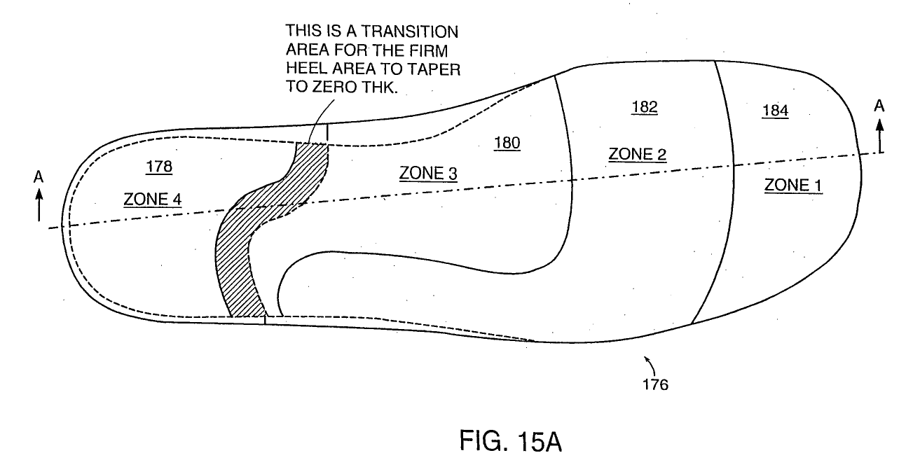

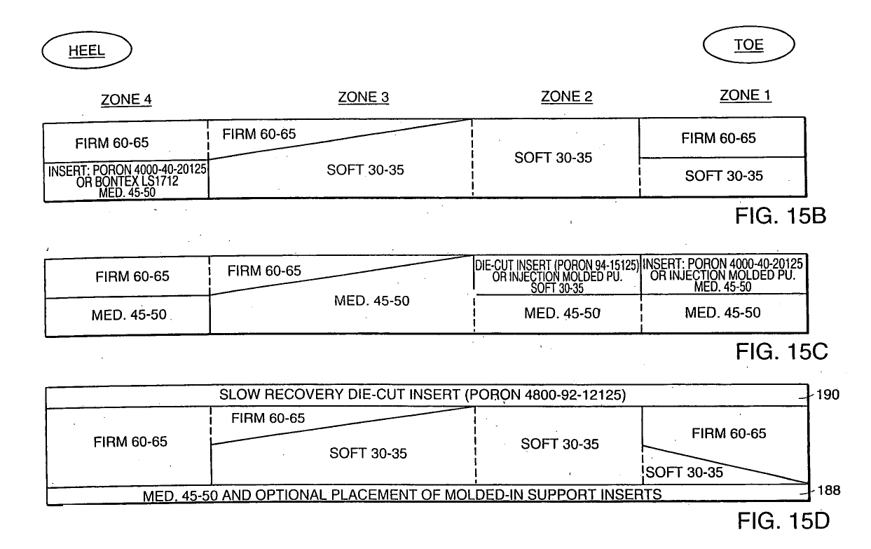

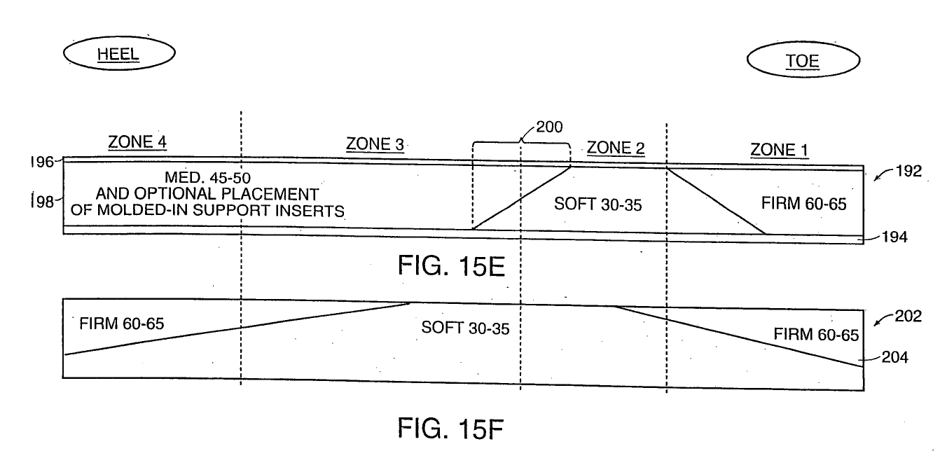

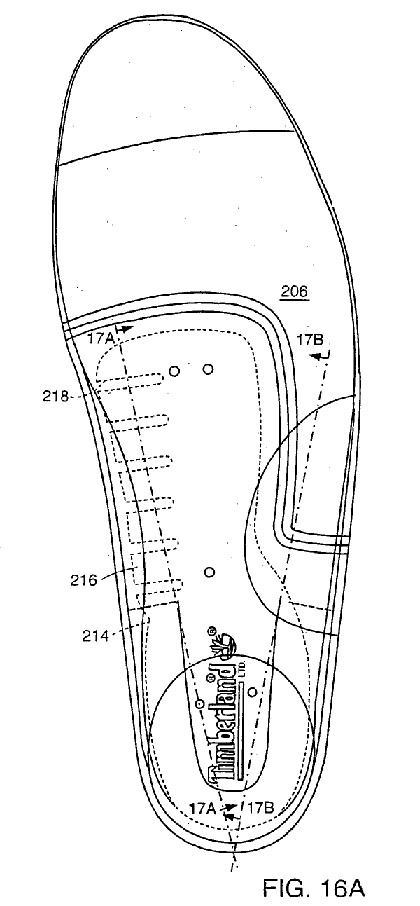

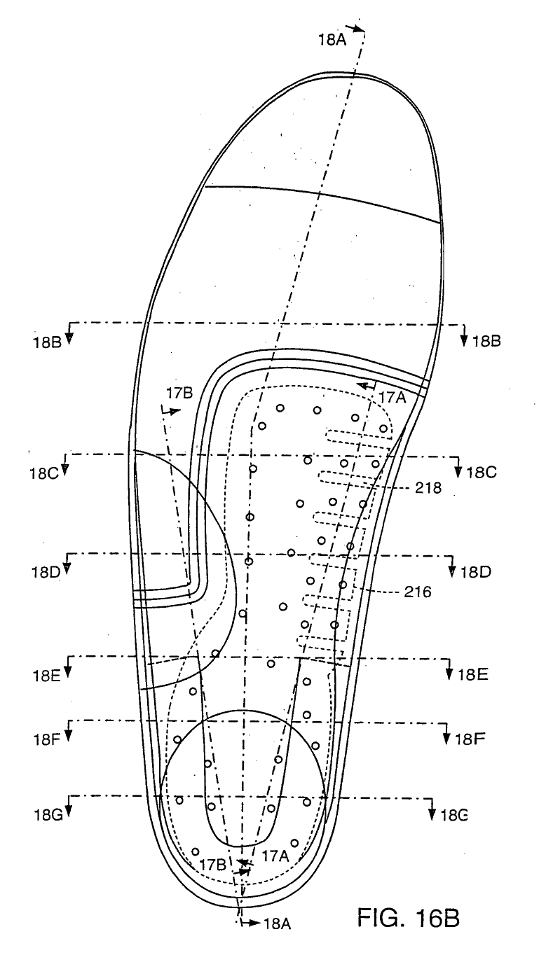

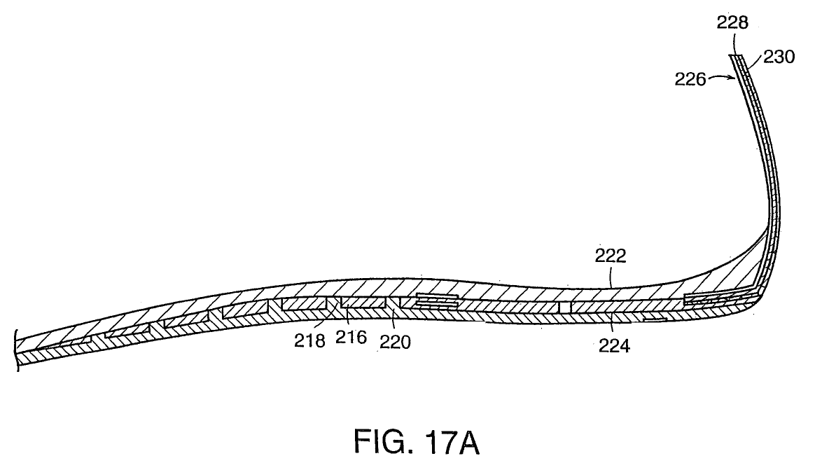

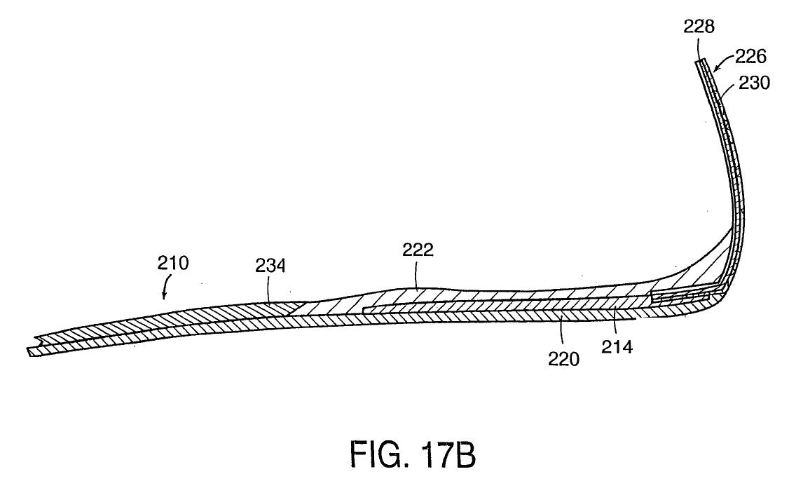

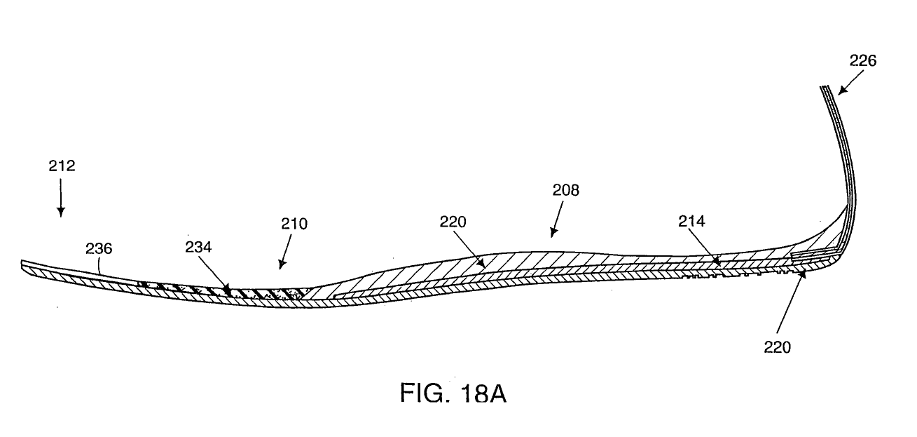

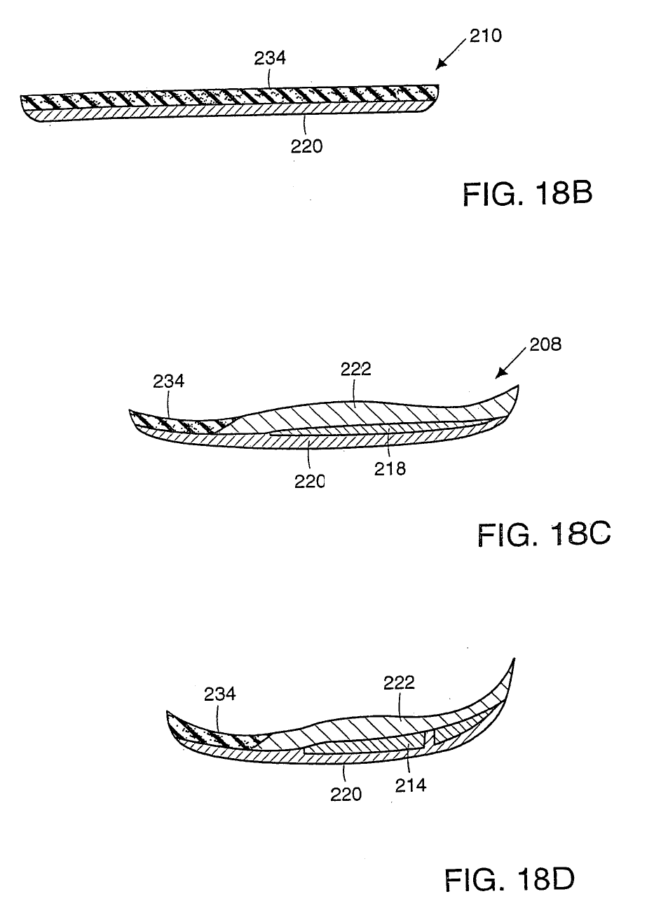

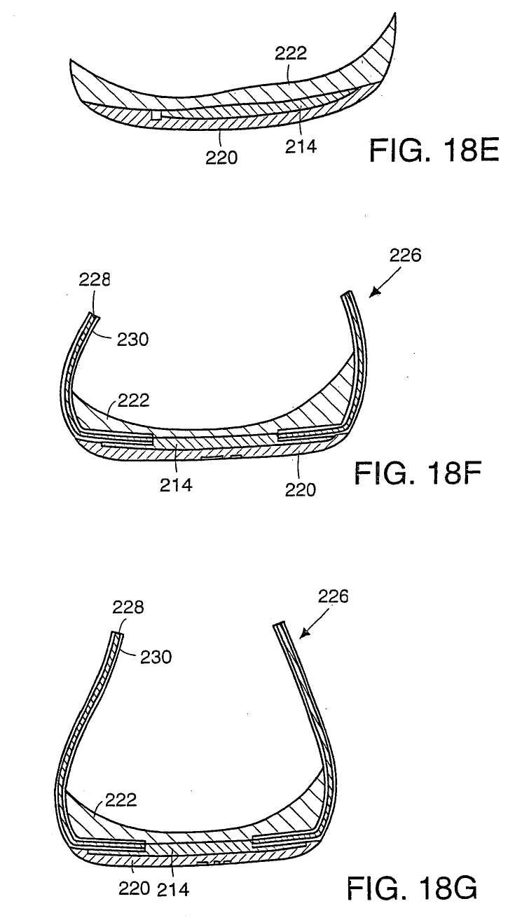

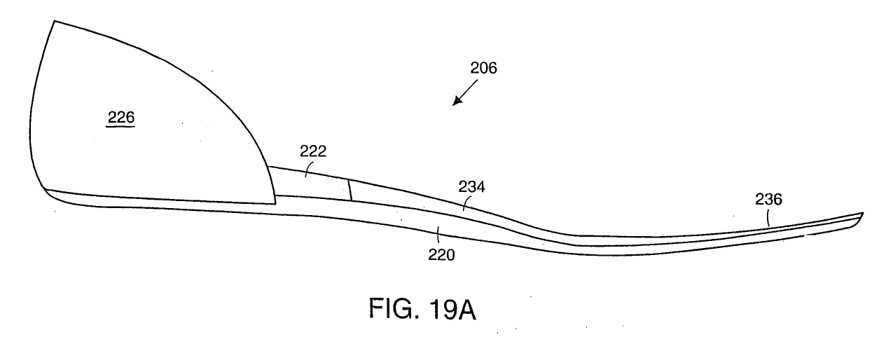

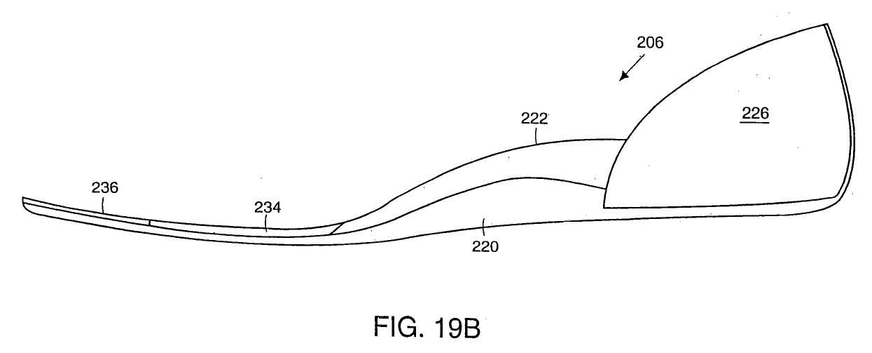

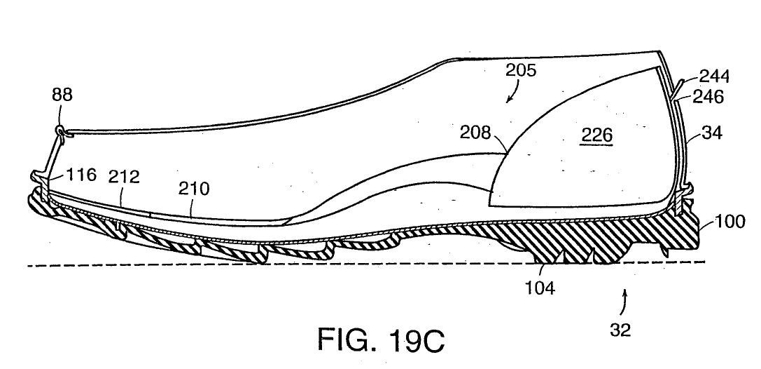

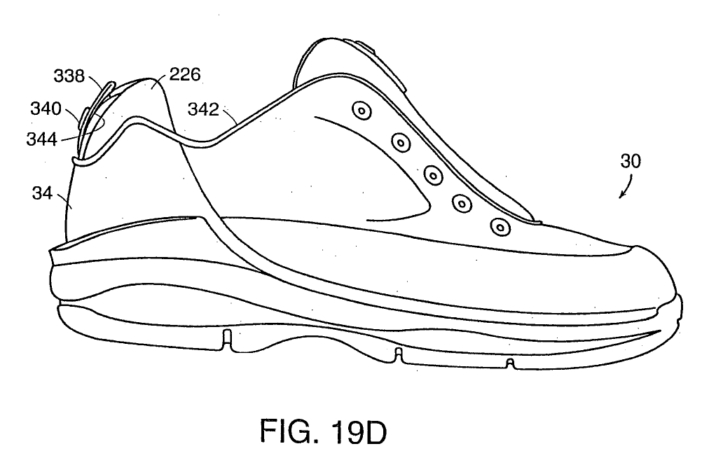

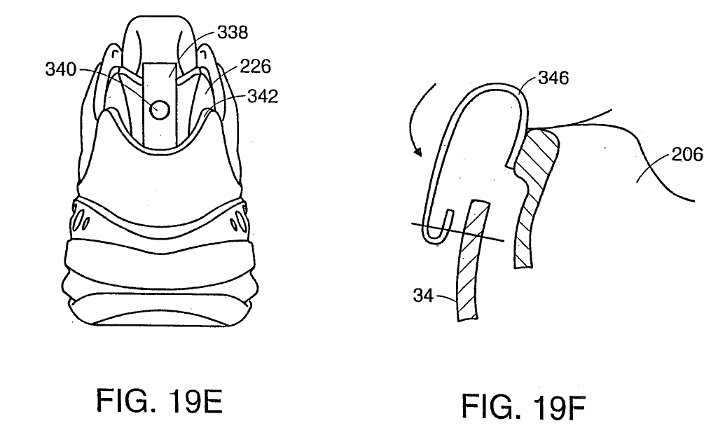

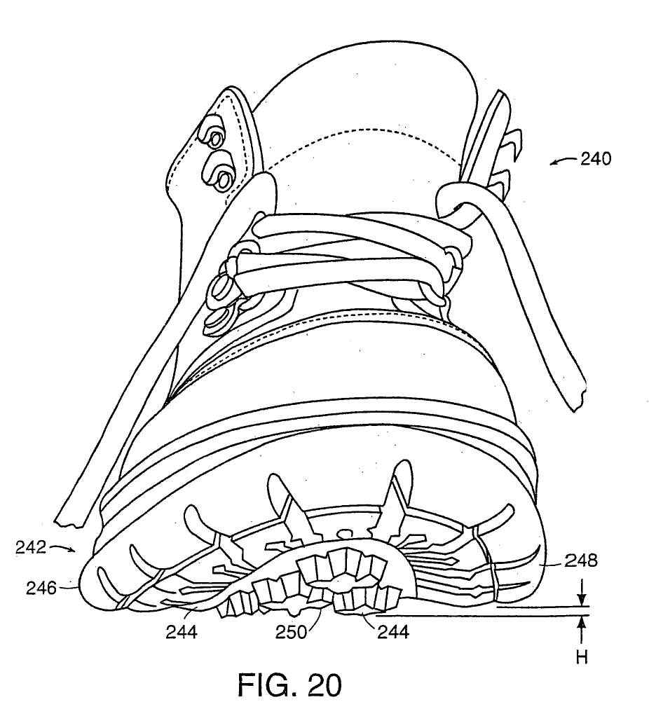

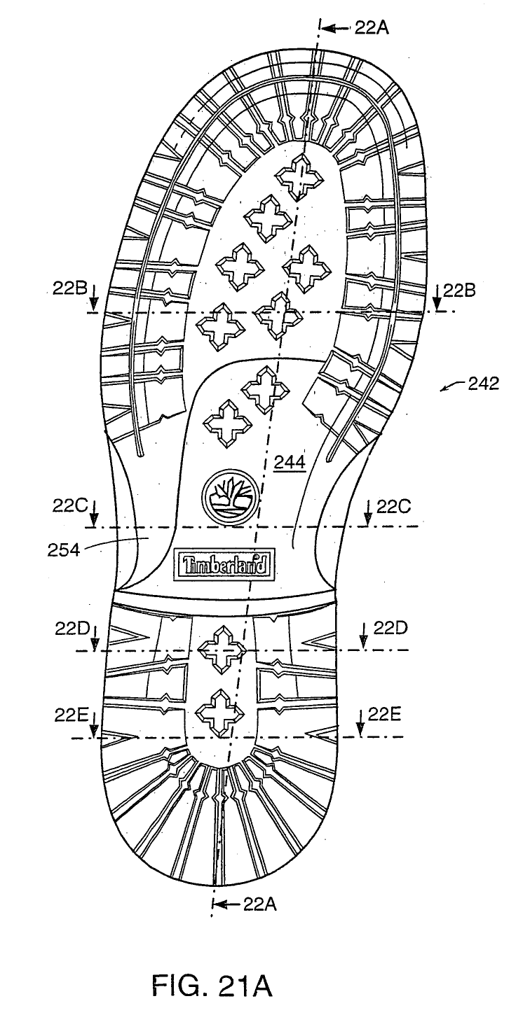

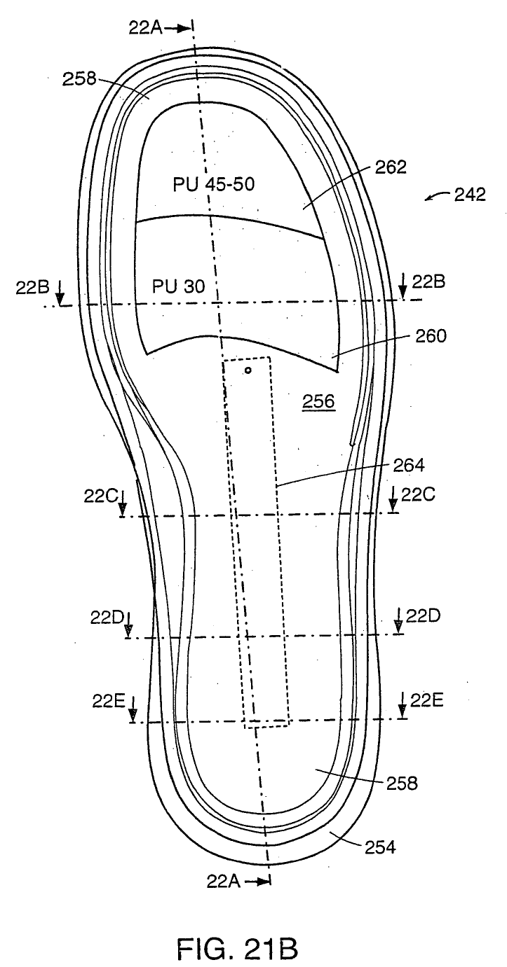

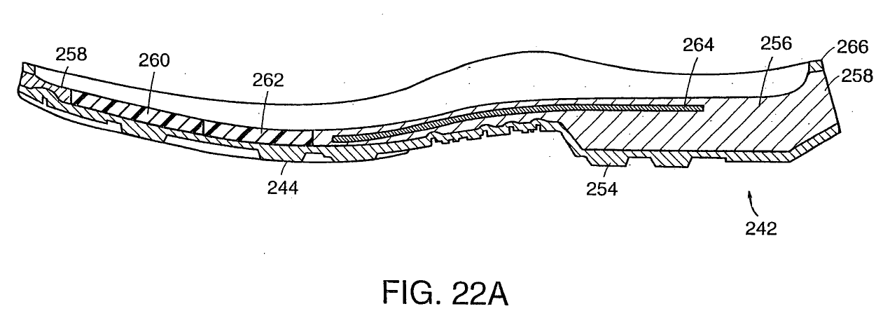

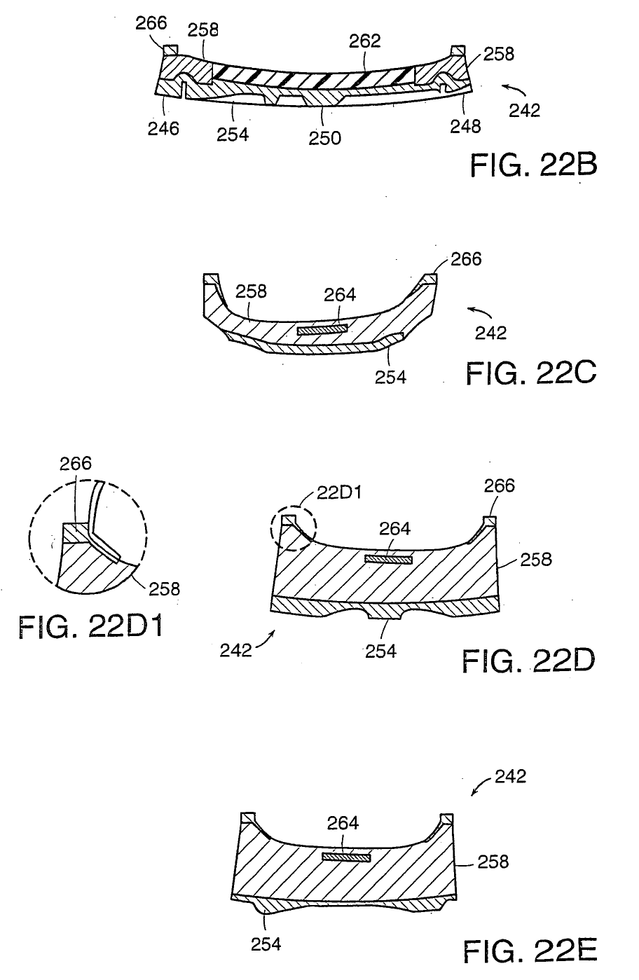

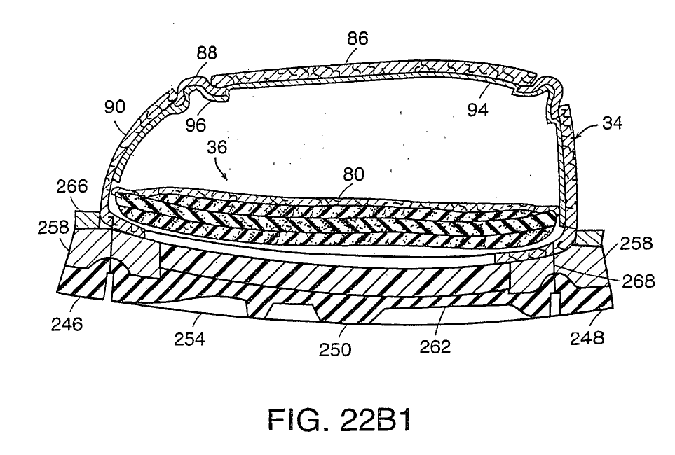

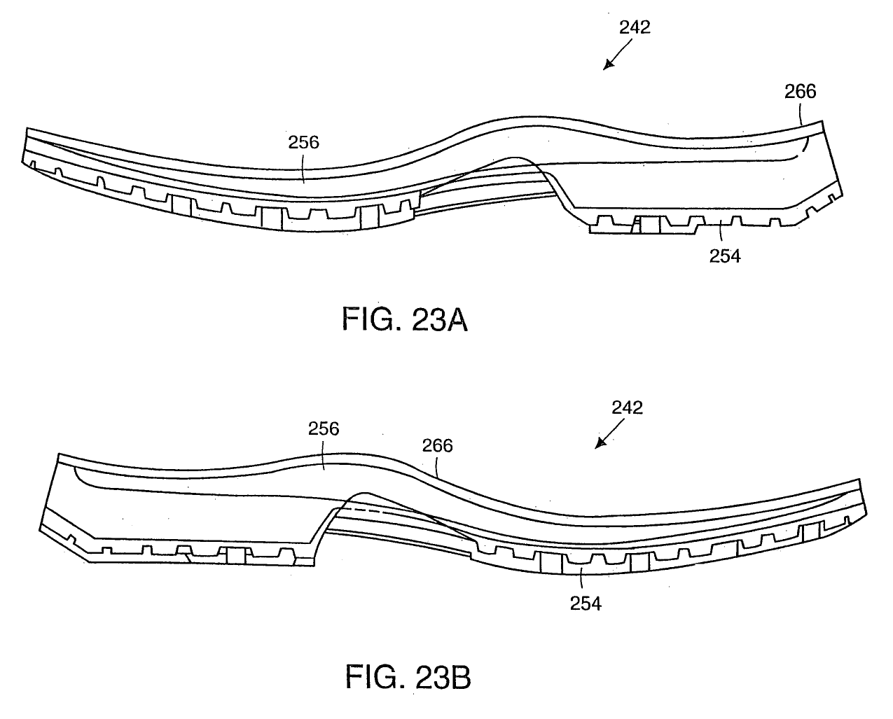

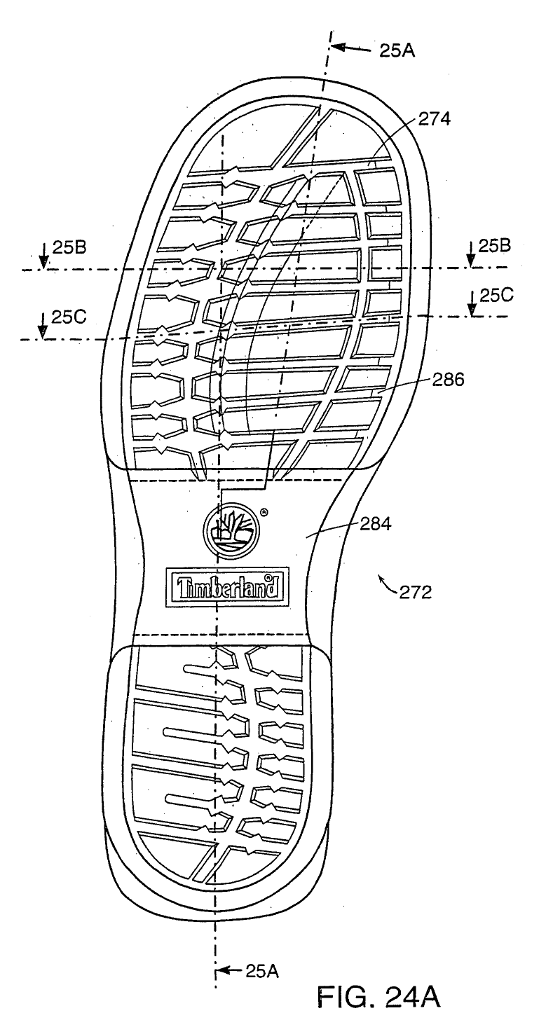

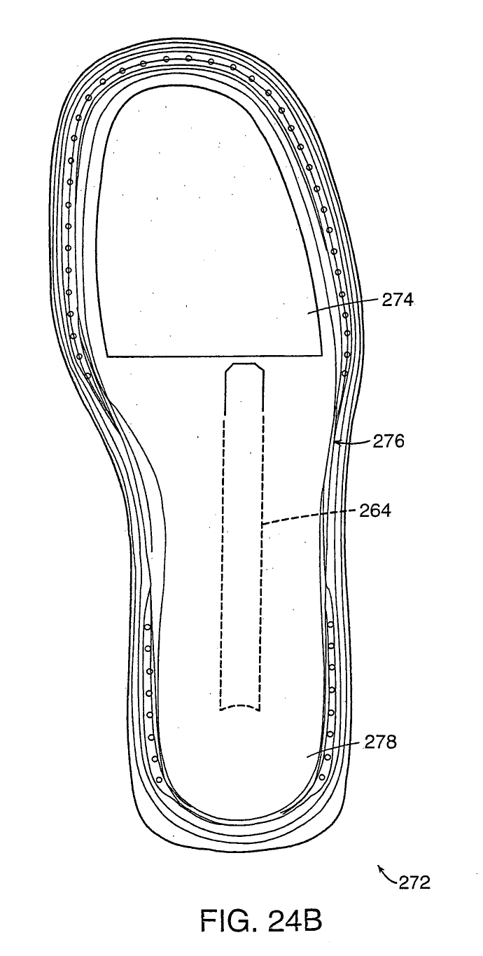

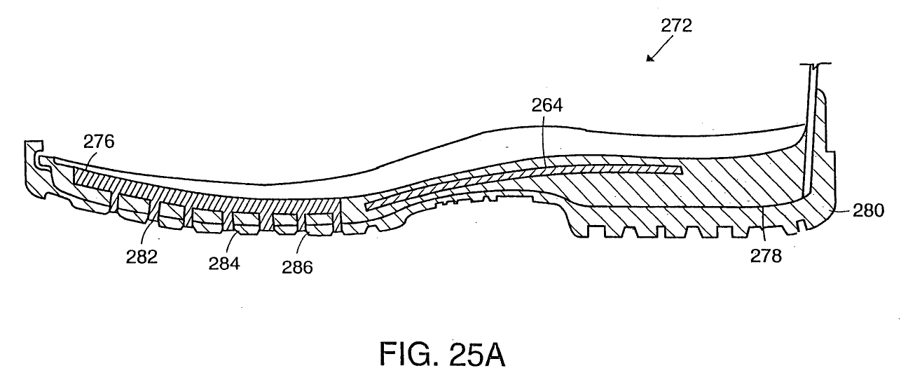

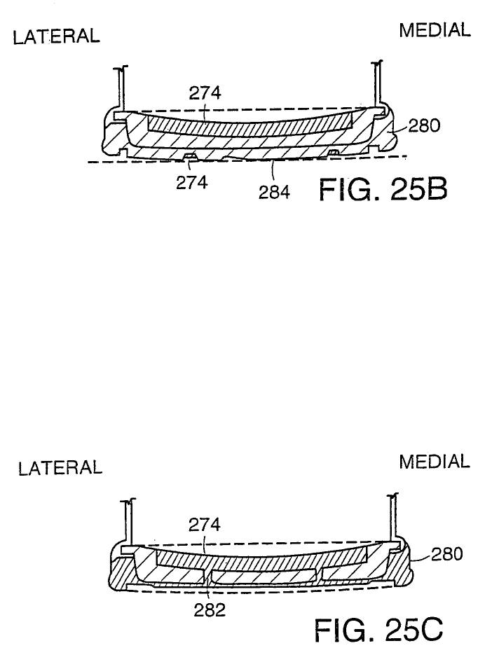

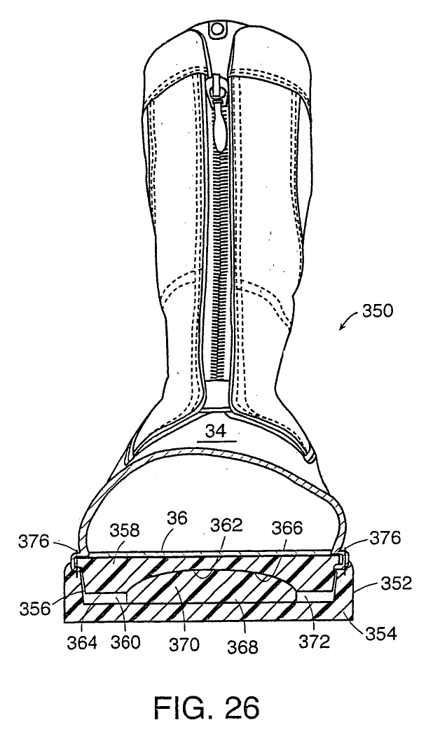

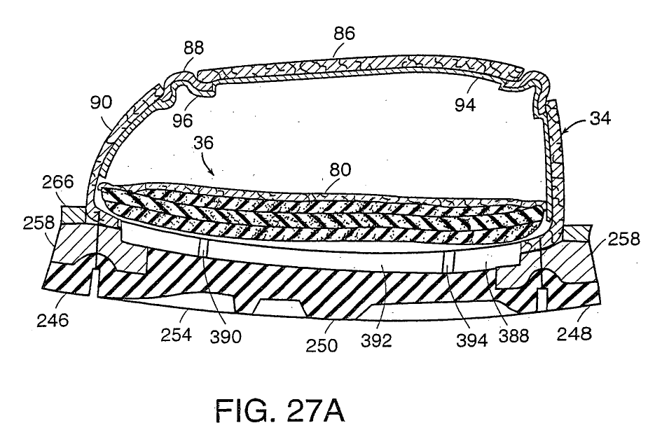

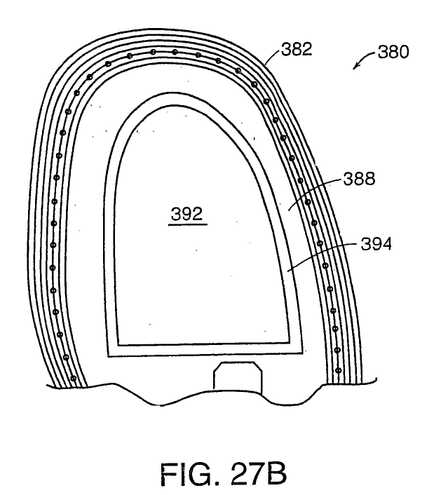

The upper 34 includes a principal portion 86 that is typically formed of leather. Portions of the principal portion 86 are joined together with seams so as to shape the upper 34. In the embodiment shown, an expansion portion 88 connects a piece of leather 86 that forms the side 90 of the upper 34 to an upper section 92 of the upper 34. The expansion portion 88 is also seen in FIG. 3. The expansion portion 88 allows the shoe to expand and recover according to the invention as explained below including related to FIGS. 11A and 11B. In addition, the upper 34 has an inner lining 94. The inner lining 94 has at least a portion that is an expansion portion 96.

Still referring to FIG. 4B, the bottom 32 of the shoe 30 has a base or outer sole 100 and an inner liner 102. The base 100 has a first material portion 104 with a series of slots or grooves 106 that are filled with a second material portion 108.

In an embodiment, the first material portion 104 is formed of an elastomer material such as rubber, thermoplastic rubber (T.P.R.), or thermoplastic urethane (TPU). The second material portion 108 is formed of a compatible or identical elastomer of a lower durometer or specific gravity such as Huntsman Avalon 65 AE TPU. The first material portion 104 and the second material portion 108 are molded together during the manufacturing process, such as a dual component injection molding or co-extrusion process.

The second material portion 108 has a “u”-shaped channel 110 that extends longitudinally to allow expansion. The inner liner 102 of the bottom 32 forms a platform for the chassis 36. The inner liner 102 has a pair of slots 112 that extend longitudinally in the shoe 30 and overlie the “u”-shaped channel 110 in the second material portion 108 of the base 100 of the bottom 32. The chassis 36, as described below in further detail, has a sturdy base layer 76; the primary purpose of the inner liner 102 of the bottom 32 is to give a finished look. The inner layer 102 in an embodiment is a pressed fiber board or a non-woven board. The pressed fiber board inner liner 102 can be manufactured from material marketed and sold under the trade name “Bonstich 305” by Bontex, Inc.

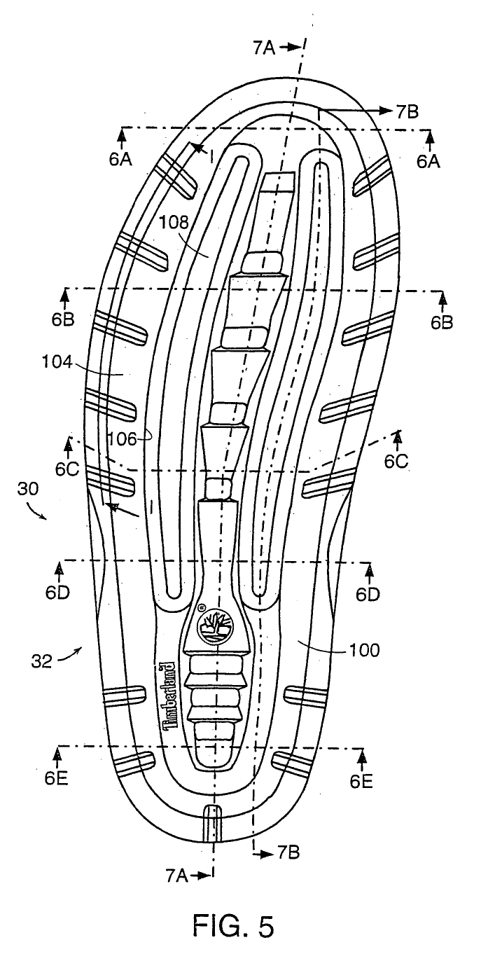

Referring to FIG. 5, a bottom view of the base 100 of the bottom 32 of the shoe 30 according to the invention is shown. The base 100 of the bottom 32 is formed of the first material portion 104 having the pair of grooves or slots 106. These grooves 106 extend generally longitudinally in the forefoot and midfoot regions of the bottom 32. The grooves 106 receive the second material portion 108, the expansion portion, which expands to widen the shoe as more force is applied to the bottom 32.

The shoe widens to accommodate the expansion of the foot. The bottom is engineered such that the expansion portion expands when the force is exerted and contracts or recovers when the force is reduced. It is recognized that the bottom can be designed to expand to a different width or to expand at different load levels through material selection and geometry. For example, children's shoes could be designed to expand less and with a different force level needed to cause expansion.

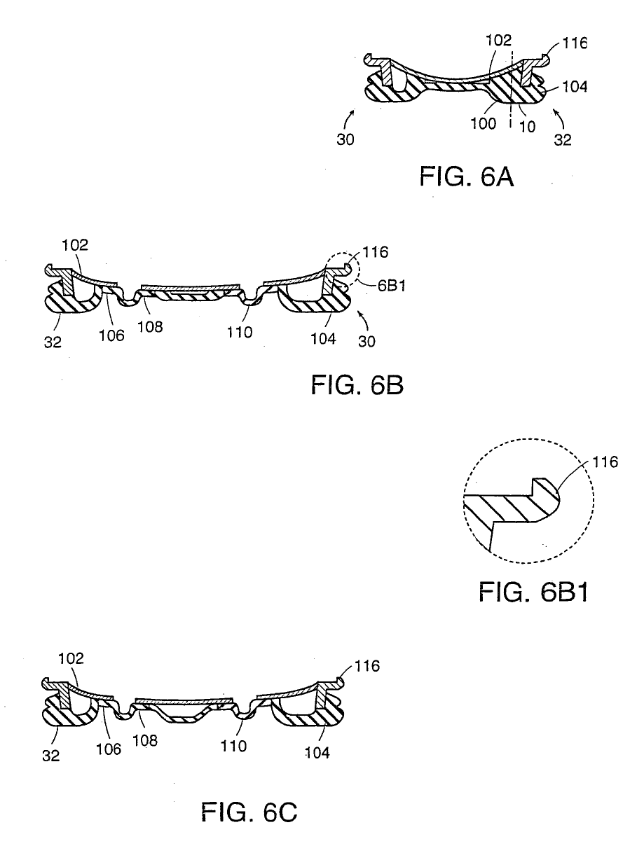

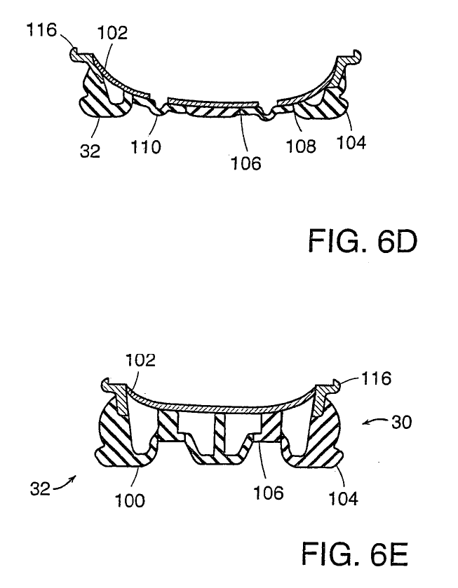

Referring to FIGS. 6A-6E, cross sectional views of the bottom 32 of the shoe 30 are shown. FIG. 6A shows a section near the tip of the shoe 30. The base 100 of the bottom 32 has the first material portion 104 extending from the medial side or edge to the lateral side or edge. At each of the edges of the bottom 32, a lip 116 projects upward which forms the welt 72 with the upper 34 as seen in FIG. 4B. In the embodiment shown, the lip is formed of the same material as the second material portion 108. The inner liner 102 is shown overlying the base 100 of the bottom 32 of the shoe 30.

FIGS. 6B-6D each show a section of the bottom 32 of the shoe 30 through the region that has an expansion portion. The base 100 has the first material portion 104 with the pair of grooves 106. The grooves 106 extend generally in a longitudinal direction as shown in FIG. 5 and receive the second material portion 108, the expansion portion. Besides the second material portion 108 being more flexible and having a lower durometer than the first material portion 104, the second material portion 108 has a geometry to facilitate expansion. The second material portion 108 of the base 100 of the bottom 32 of the shoe 30 has the “u”-shaped channel 110 which can flatten to allow expansion.

The inner liner 102 is shown overlying the base 100 of the bottom 32 of the shoe 30 in FIG. 6B. The inner liner 102 has a slot 112 overlying each of the “u”-shaped channels 110 on the base 100 to allow the edges of the inner liner 102 to move apart as the base 100 expands.

The lip 116, which extends completely around the bottom 32, and thus seen in FIGS. 6B-6E, projects upward from the base 100 to create a location to which the upper 34 is sewn to form the welt 72. An enlarged section of the lip 116 is seen in FIG. 6B1. The lip 116 has a groove or slot to facilitate attachment of the upper. While the connection of the upper 34 to the bottom 32 is shown with the upper 34 overlying the lip 116 in FIG. 6B1, it is recognized that other methods can be used to secure the upper 34 to the bottom 32. FIG. 22B1, which is discussed below, shows another arrangement of securing the upper 34 to the bottom 32. In addition, other methods of attachment include sewing the upper to the bottom in a portion that is underlying the chassis 36. In addition to sewing, other methods of attachment include gluing the upper 34 to the bottom 32 of the shoe 30 or the molding of the bottom 32 to the upper 34 of the shoe 30.

FIG. 6E shows a section in the heel region of the bottom 32 of the shoe 30. As will become clearer below, it is not desirable in this embodiment to have the heel region of the bottom 32 expand. The heel of the foot is retained to maximize the natural cushioning ability of the heel by keeping the fat pads under the heel bone for cushioning. While the base 100 has both the first material portion 104 and the second material portion 108, the second material portion 108 is not designed to expand in this zone. In contrast to what is shown in FIGS. 6B-6D, the second material portion 108 has a thick cross-section and not the geometry to flatten therein allowing expansion. The longitudinally extending sidewalls of the grooves 106 can be formed at a downward outwardly directed angle that is sufficient to cause lateral widening or spreading of the grooves 106 and bottom 32 when the toe portion of the bottom 32 is bent or flexed upwardly during walking or running. The grooves 106 and bottom 32 then contract when the bottom 32 straightens or returns to an unflexed state. As a result, lateral expansion and contraction of the shoe can occur in response to the flexure and return of the bottom 32. In addition, bottom 32 can have a generally central, downwardly facing, longitudinally extending crown on the sole 100, such as that shown in FIGS. 22B1 and 27A. The crowned center of the sole 100 extends downwardly below the side edges. The crowned sole 100 flexes and flattens when the sole 100 contacts the ground during walking or running, causing lateral expansion of the bottom 32.

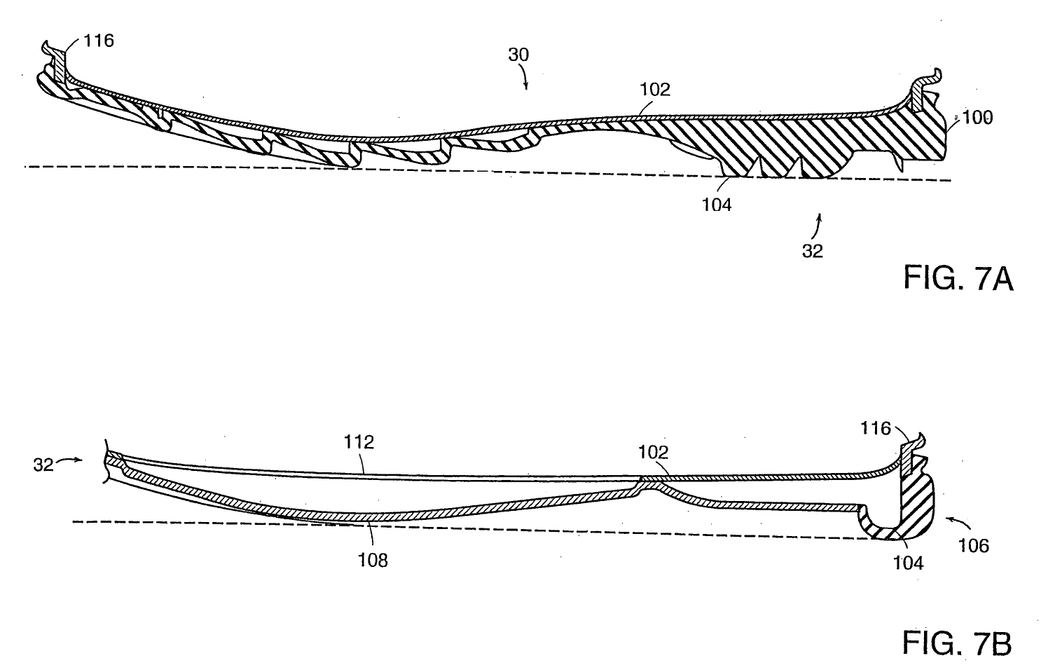

FIG. 7A is a sectional view taken generally along the center line of the bottom 32 of the shoe 30. The section of the base 100 shown is the first material portion 104. In the forefoot and the midfoot regions, the portion shown is the first material portion 104 between the second material portion 108. Overlying the base 100 is the inner liner 102. The lip 116 for attaching the upper 34 is shown at the front and back of the shoe 30.

FIG. 7B is a sectional view going through the expansion portion, the second material portion 108. As indicated above, while the heel region includes portions that are formed of the second material 108, the geometry and the thickness of the second material portion 108, such as seen in FIG. 6E, result in the heel not expanding as described above with respect to the metatarsal region. The section through the inner liner 102 includes the slot 112 in the forward part of the bottom 32 that overlies the “u”-shaped channel 110.

As indicated above, as force is applied to the bottom 32 of the shoe 30 and the foot 40 expands in the metatarsal 50 region, the base 100 expands by stretching and flexing the second material portion 108 and in particular the “u”-shaped channel 110. The inner liner 102 does not physically expand, but has the slots 112 as best seen in FIGS. 6B-6D that allow the edges to move apart.

The bottom 32 of the shoe 30 can include a shock diffusion plate. The shock diffusion plate overlies the base 100 of the bottom 32 in the heel zone for diffusing the shock of the heel of the shoe hitting the ground. A shock diffusion plate is further described in U.S. Pat. No. 6,205,683, the contents of which is incorporated herein by reference in its entirety.

While the bottom 32 of the shoe 30 expands, the chassis 36 serves a different purpose of properly supporting different regions of the foot 40. The chassis 36 has contours to better support and retain “fat pads” under the boney structure for cushioning. The chassis has a plurality of zones. Each zone is specially designed or tailored by selection of materials and shapes to the portion of the foot that overlies and contacts that zone of the chassis. The chassis manages and spreads the force the foot receives as the foot is dynamically loaded as the foot moves.

Referring to FIGS. 8A and 8B, the top and the bottom views of the chassis 36 are shown. As indicated above with respect to FIG. 2, the foot 40 has various regions including the heel 46, the metatarsal region 50, and the toe region 44. In the embodiment shown in FIGS. 8A-10B, the chassis 36 has a plurality of regions or zones wherein the zones are tailored to reflect the region of the foot 40 that overlies and contacts the chassis. The chassis 36 of the embodiment shown has three zones or regions; a heel zone 120, a metatarsal zone 122, and a toe zone 124, as seen in FIG. 8A. While referred to as the metatarsal zone 122, the metatarsal zone 122 underlies the MP joints 52 and the first metatarsal and not the second through fifth metatarsals in typical embodiments.

FIG. 8B shows the bottom or base layer 76 of the chassis 36, the portion that contacts the inner liner 102 of the bottom 32 as seen in FIG. 4. The bottom of the chassis 36 has a plurality of grooves 126. The purpose of the grooves 126 is to allow for greater flexure of the chassis 36. The location of the grooves 126 relative to the edges of the zones 120, 122, and 124 is identical because the grooves delineate the zones. The bottom or base layer 76 of the chassis 36 is formed of a single material in this embodiment. The grooves 126 while adding flexure to the chassis 36 also can be employed to denote zones.

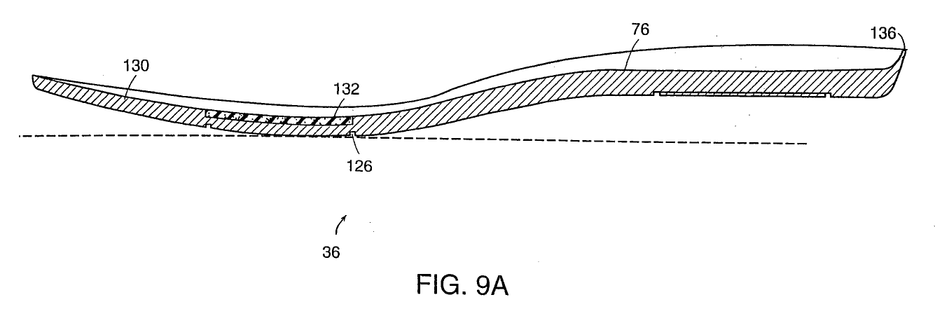

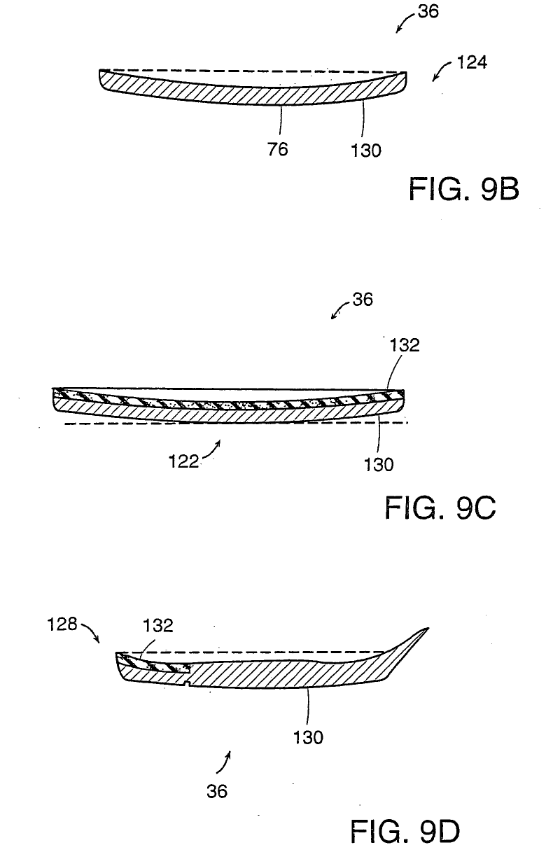

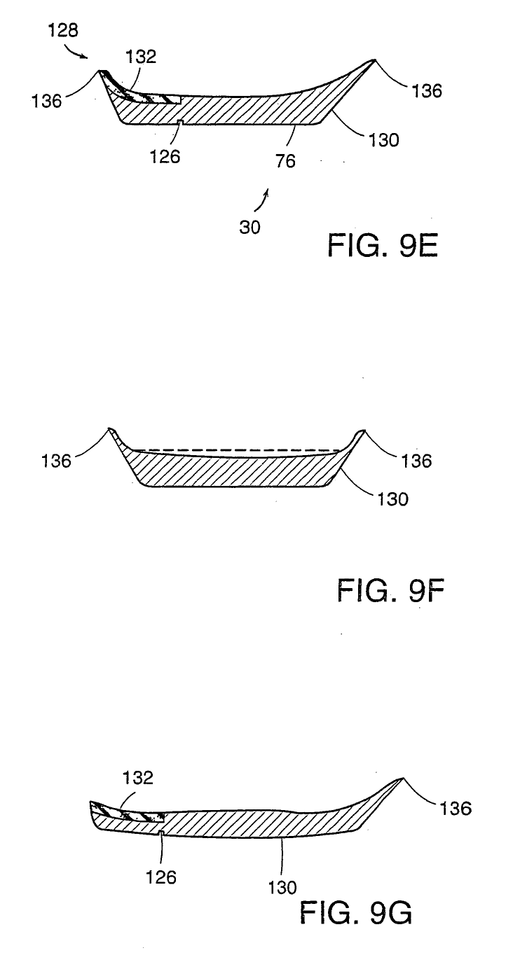

FIG. 9A is a sectional view taken generally along the longitudinal center line of the chassis 36 of the shoe 30. The chassis 36 has the base layer 76. The base layer 76 extends through to the top in the heel zone 120 and the toe zone 124. The material 130 for the base is selected to be resilient for the heel zone 124. In the metatarsal zone 122, a second material, an inlay 132 overlies the base layer 76. The inlay 132 is selected to be a cushion material for the metatarsals 50 of the foot 40. The grooves 126 for flexure are shown.

FIGS. 9B-9G are sectional views taken along their respective lines in FIG. 8B. In the heel zone 120 and the toe zone 124, the chassis 36 has the base layer 76 of the material 130 extending from the bottom to the top, as is seen in FIG. 7F and FIG. 9B respectively. It will be noted that the toe zone 124 has a curved shape as seen in FIG. 9B in contrast to the flat bottom of the heel zone 120 as seen in FIG. 9F. The curved shape of the chassis 36 works in conjunction with the expansion of the bottom 32 of the shoe 30 to allow the edges to move apart when loaded as discussed above.

Similarly, referring to FIGS. 9C, 9D, and 9G, the sections are not planar but have a curved shape such that when pressure is applied to the chassis 36, similar to the bottom 32, the chassis 36 flattens therein increasing the width of the shoe.

In addition, as seen in FIGS. 9C, 9D, and 9G, the inlay 132 for the metatarsal zone 122 overlies the base layer 76 to provide a different material for this region. The inlay 132 in the embodiment shown extends into an arch zone or region 128 as seen in FIG. 9D and best seen in FIG. 8A.

FIG. 9E shows the inlay 132 overlying the base layer 76 in the arch zone 128. In contrast to FIGS. 9D and 9G, the base layer 76 in FIG. 9E is planar since this portion of the shoe 30 does not expand. It is desirable that the shoe not expand in this region, the heel zone 120, because it is desired that the heel of the foot be retained in position to maximize the natural cushioning ability of the heel by keeping the fat pads under the heel bone.

The chassis 36 has an upper edge contour 136 in the arch region 128 and the heel zone 120 for support. In the heel zone 120, the curved upper edge 136 is on both the medial and lateral sides as seen in FIG. 9F. The curved upper edge 136 decreases in height or levels out moving from the rear towards the front on the chassis 36 and more drastically on the lateral side than the medial side as seen in FIGS. 9D, 9E, and 9G. The curved upper edges give lateral support to the foot and keep fats pads under the bones of the foot for cushioning.

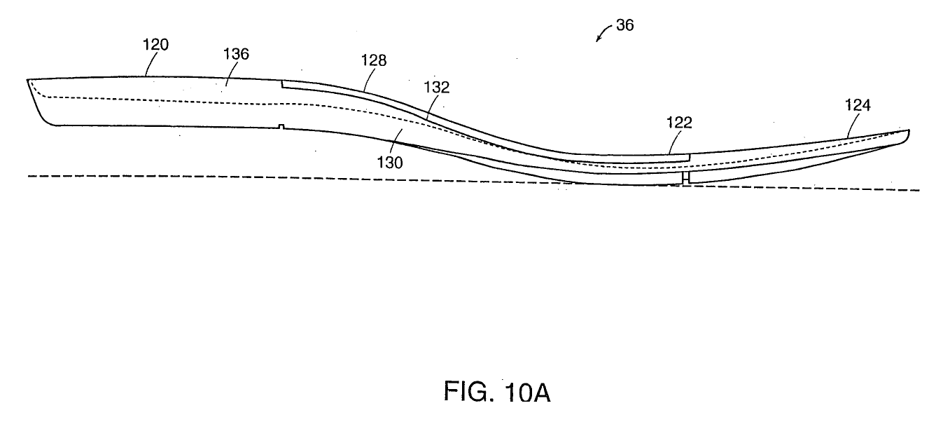

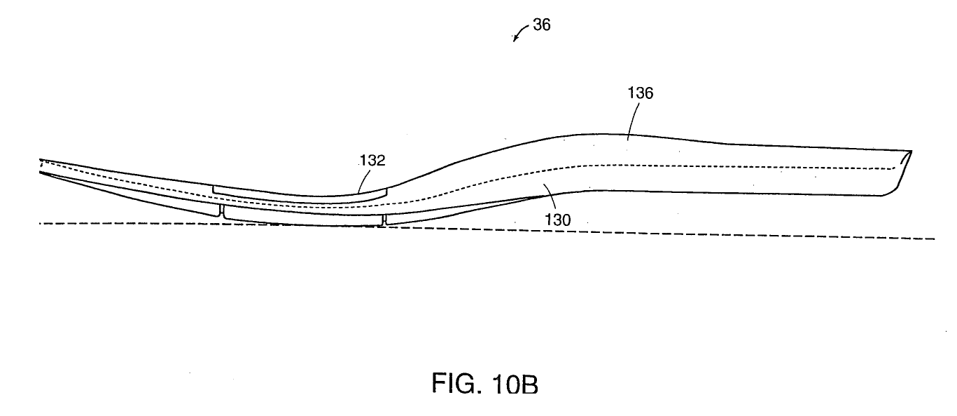

FIG. 10A shows the lateral side view of the chassis 36 of FIGS. 8A-9G. FIG. 10B shows the medial side view. As indicated above, the inlay 132 overlies the base layer 76 in the metatarsal zone 122 and on the medial side extends back towards the heel in the arch zone 128. The curved shape of the chassis 36 is evident in the toe zone 124 and the metatarsal zone 122. Likewise, the curved upper edge 136 of the heel zone 120 is shown.

With the bottom 32 and the chassis 36 of one embodiment of the shoe 30 discussed, the upper 34 is described in greater detail below. Referring back to FIG. 4, while leather typically expands during the useful life of the footwear, the shoe 30 has portions, the expansion portion 88, that expand and recover back to their previous state as the foot 40 moves to provide both comfort and support for the foot 40. In addition, the shoe 30 has a more tailored look than for example a Euro Comfort.

Referring to FIG. 11A, an enlarged section of the upper 34 is shown with an expandable omega seam 140 as the expansion portion 88. The omega seam 140 is sewn to the two pieces of material, the side 90 and the upper section 92 of the upper 34 by a pair of lines of stitching 142. When the foot expands, the upper section 92 of the upper 34 deflects upward and the omega seam 140 stretches to a more planar state as shown in FIG. 11B.

FIG. 12A is a sectional view of a segment of the upper 34 showing an alternative expansion portion connecting the side 90 and the upper section 92 of the upper 34. The expansion portion is a piece of goring 144, an expandable material. The two pieces of material 90 and 92 are brought in proximity to each other. The goring 144 underlies the two materials, the side 90 and the upper section 92. The goring 144 is sewn using lines of stitching 142 to secure both pieces of material.

Referring to FIG. 12B, when the foot expands, the goring 144 stretches to expand with the foot. Likewise when the foot contracts, the goring 144 contracts to its original dimension bringing the two pieces of the material 90 and 92 of the upper 34 in proximity to each other holding the foot in a comfortably snug position.

With respect to the upper 34 of the shoe 30, the goring 144 of seams or the expandable omega seam 140 allows for stability of the foot, while also allowing for expansion and contraction of the upper 34 as the foot moves. An alternative to the goring 144 of the seams or the expandable omega seam 140 is the use of materials such as a stretch leather, neoprene, lycra, spandex, or any appropriate stretchable material which allows a give and take in certain directions but retains the foot in other directions.

While the shoe 30 shown in FIG. 3 has an inner lining 94 on the upper 34, it is sometimes desirable to have a liner which is a waterproof liner which encircles the foot 40 to a certain height to protect the foot 40 in wet environments. As indicated above, the shoe 30 expands and contracts in the metatarsal zone 122. The waterproof, breathable liner needs to expand and contract with the rest of the shoe for maximum comfort. In addition to being waterproof, the liner is breathable to allow the foot to breathe and allow moisture, such as perspiration, to pass from the shoe.

A side view of a waterproof, breathable liner 150 for the shoe 30 is shown in FIG. 13A. The waterproof, breathable liner 150 in the embodiment shown has a stretchable region 152 and a non-stretchable region 154. In a preferred embodiment, both regions are made of a waterproof, breathable membrane that allows the foot to breathe by allowing moisture out, but does not allow moisture or water in. The waterproof, breathable membrane can include microporous, hydrophilic, or other similar base materials promoting the passage of moisture vapor and/or low pressure air only in one direction.

It is typical in shoe construction to use a form which is referred to as a last 156 around which the upper 34 is formed and secured to the bottom 32. The waterproof, breathable liner 150 is shown on the last 156 in FIG. 13A.

Still referring to FIG. 13A, both the stretchable region 152 and the non-stretchable region 154 of the liner 150 are formed of a series of panels 158a, 158b, 160a, and 160b. The two panels 158a and 158b of the stretchable region 152 are sewn together. The stretchable region 152 is likewise sewn to the non-stretchable region 154 as seen in FIG. 13B where the panel 158a is shown sewn to panel 160c.

While the panels 158a, 158b, 160a, and 160b overlie the last 156, the panels are not secured to the last 156. The sewing of a liner, such as a conventional non-stretchable liner, in this manner is typically called a strobel last construction. The construction described here is not a conventional strobel last construction as described below. The seams where the panels 158a, 158b, 160a, and 160b of the waterproof, breathable liner 150 are joined are taped using a waterproof tape 166. The stitching is shown in hidden line under the waterproof tape in FIG. 13C. The tape can have similar breathable and stretch attributes to the membrane but the primary function is to create a waterproof seal over stitched seams and reinforce the mechanical interlock of the otherwise separate bootie panels.

In the construction method shown in FIGS. 13A-13D, the panels 158a, 158b, 160a, and 160b are sewn together prior to being placed over the last 156.

With the panels 158a, 158b, 160a, and 160b sewn together and the seams taped together, a gasket 164 is secured by adhesive to the non-stretchable region 154 in both panels 160a and 160b, and the aft portion of the stretchable region 152, in both panels 158a and 158b, as seen in FIGS. 13C and 13D. The gasket 164 is non-stretchable and underlies the heel, which in the shoe 30 is a non-stretchable region. The gasket 164 is an impermeable material. The gasket 164 is used instead of a non-stretchable membrane liner because of several factors including no need for breathability when it abuts impermeable surfaces such as nylon, TPU, or EVA and the combination of the adhesive structure for bond and barrier results in manufacturing efficiencies.

The panels 158a, 158b, 160a, and 160b do not surround the bottom of the last 156 completely. The gasket 164 completes the bottom covering of the last 156. The folding of a panel, such as panel 160a, over the last 156 and securing another panel, such as gasket 164, with an adhesive or cement is typically referred to as a cement last construction. The combining of strobel last construction and cement last construction as described with respect to FIGS. 13A-13D is referred herein as advanced combination construction (ACC). U.S. Pat. No. 6,205,683 describes a strobel stitched and cement construction.

Before the gasket 164 is secured to the waterproof, breathable liner 150, the seams where the panels 158a, 158b, 160a, and 160b are taped using a waterproof tape 166 are shown in FIG. 13D. The tape can have similar breathable and stretch attributes as the membrane but the primary function is to create a waterproof seal over stitched seams and reinforce the mechanical interlock of the otherwise separate liner or bootie panels.

In an embodiment, the gasket 164 is made of a material having uniform directional strength that is resistant to flex cracking over a wide temperature range (e.g., −13° F. (−25° C.) to 1001F (38° C.)). The gasket 164 also is capable of remaining water impermeable both from a bond retention strength and product integrity after extended angle flexing (e.g., 100,000 45° flexes) in a high temperature/humidity environment (e.g., 120° F. (49° C.) 98% relative humidity typically).

An alternative construction of a waterproof, breathable liner 168 is shown in FIG. 13E. The waterproof, breathable liner 168 has five panels, 160a, 160b, 160c, 160d, and 158a. The liner 168 has four non-stretchable panels 160a, 160b, 160c, and 160d that extend from the toe to the heel to form a non-stretchable region 154. A stretchable panel 158a forms a stretchable region 152 which overlies the foot in front of the ankle, generally underlying where a tongue of a shoe is located. The waterproof liner can have a gasket as described above or a gasket which completely underlies the foot as described with respect to FIGS. 14C and 14D. It is recognized that portions of the breathable liner can underlie the entire foot.

An alternative waterproof, breathable liner 170 is shown in FIGS. 14A and 14B. The waterproof, breathable liner 170 has a series of panels including a stretchable region 172 having three panels 158a, 158b, and 158c and a non-stretchable region 174 having a pair of panels 160a and 160b. The panels 158a, 158b, 158c, 160a, and 160b or bottomless bootie parts are assembled by closed seam or butting edges together and using a zig zag, merrow, or similar stitch. The stitched seams are sealed using a waterproof adhesive system applied manually or via dispensing system (such as a heat activated taping 166).

During manufacture of the footwear or shoe 30, the waterproof, breathable liner 170 is attached to the upper 34 either as the upper 34 is formed, or inserted into the upper 34 after the upper 34 is formed. In either case the liner 170 is attached above the highest desired waterproof point, typically by sewing, and attached at other portions, typically by adhesive such as in the toe region, so that the liner does not move or shift relative to the upper 34 and expands and recovers with the upper. The chassis 36 overlies the bottom of waterproof liner and/or gasket to assist in keeping the liner in position.

The waterproof, breathable liner 174 is placed over a last or form 156 prior to the forming of the upper 34 around the last or with the upper 34. The last or form 156 can have an insole or chassis 398 temporarily attached to facilitate shaping the waterproof, breathable liner 174.

As indicated above, the liner is partially formed using a cement construction. In the embodiment shown in FIGS. 14A and 14B, the fore portion of the bottom of the liner is constructed using a strobel construction. The aft portion of the bottom of the liner is constructed using a cement last construction. In the cement last construction portion an adhesive band is applied to the inner lining of the waterproof, breathable liner 170 and the insole 398. The liner has a “lasting allowance” to allow the liner to lay flush. In a typical embodiment, the allowance is approximately 0.75″ (19 mm) from the last feather edge. The waterproof liner is folded around the last or form 156 and into contact with the insole 398. While drawing the waterproof, breathable liner 170 to the last or form 156, the waterproof, breathable liner 170 or the insole 398 material is massaged to minimize gathering or wrinkling in any concentrated area. (i.e., the gathering of the material is uniform or flush, not bunched up non-uniformly such as in one area.) Ideally the last or form 156 or the insole 398 bottom can be premarked to include a reference point for properly positioning the waterproof, breathable liner 170 or bottomless bootie when drawn against the last or form 156 or the insole 398.

Referring to FIG. 14B, after the waterproof, breathable liner 170 is secured to the insole 398 as seen in FIG. 14A, a gasket 164 is secured to the heel region. As in the previous embodiments, the seams that are sewn are taped before the gasket 164 is secured. The tape is not shown in FIG. 14B for clarity.

While the embodiments above describe a non-stretchable gasket, it is recognized that in certain situations a stretchable gasket may be preferred and used, for example, where the gasket covers the entire bottom. FIGS. 14C and 14D show a construction of a waterproof, breathable liner 300 having a full gasket bottom. FIG. 14C shows the waterproof, breathable liner 300 with a non-waterproof stretchable panel or material 302 stitched to the forward portion of the bottom to the panels 304a and 304b of the waterproof liner to hold the pattern together. Stretch panels (including the non-waterproof insert) are inserted into the pattern to facilitate 360° expansion and contraction as required. A bottom gasket 308 is sized to meet the feather edge (typically 5 mm beyond the outer stitch seam edge). By the gasket 308 extending over certain seams, those seams do not need to be taped in addition. Those seams that are not covered by the gasket 308 are taped prior to the application of the gasket 308; the tape is not shown in FIG. 14D for clarity.

An alternative method of construction of a waterproof, breathable liner 314 is shown in FIG. 14E. The waterproof, breathable 314 liner is shown in FIG. 14E. The waterproof, breathable liner 314 is formed of one or more panels of either or both stretchable material 316 and non-stretchable material 318 forming stretchable and non-stretchable regions 316 and 318. Similar to the previous embodiment, the panels 320a, 320b, 322a and 322b do not cover the entire bottom. Rather, as best seen in FIG. 14F, the panels 320a, 320b, 322a and 322b have a layer 324 folded over forming a channel 326 to receive a string or cord 328. The bottomless waterproof, breathable liner 314 is placed over a last or form 156. The last 156 can have an insole or chassis 330. The cord 328 is pulled to uniformly gather the bottomless waterproof, breathable liner 314 around the last 156. Similar to the cement last construction, the material can be massaged to minimize gathering or wrinkling in any concentrated area.

When the bottomless waterproof, breathable liner 314 is uniformly drawn against the last or form 156 with or without an insole 330, the cord 328 is tied off either against the start end or to pins molded on the last or form 156 or molded or attached to the insole 330 above. Ideally the last or form 156 or insole 330 bottom would be premarked to include a reference point for a properly positioned bottomless bootie when drawn against the last or form 156 or insole 330. Once drawn against the last or form 156 or insole 330 and properly positioned and secured, the application of a waterproof gasket material 332 is typically required to make the footwear waterproof. But in some embodiments, the bottom 32 can be direct injected or vulcanized to the waterproof, breathable liner 314 and likely the upper 34, therein creating a waterproof bottom 32 and not requiring a gasket 332.

It is recognized that other techniques for assembling the waterproof, breathable liner can be used. One of the factors for determining what style of waterproof, breathable liner is used is the design of the shoe. For example, one criteria is the location of the stretch and recovery portions of the upper. It may be desirable to place non-stretch material in the toe section of the certain patterns to retain the shape, such as seen in FIG. 13E.

In several of the embodiments disclosed, the waterproof, breathable liner has a moisture vapor permeability rating greater than or equal to 3.6 mg/cm²/hr measured according to SATRA PM47 and be resistant to or have a protective outer layer providing abrasion and puncture resistance and minimize tearing due to stretch when saturated and/or cemented to the outer upper material. In certain embodiments, the liner can be laminated to foams, insulators and/or lining materials to form a waterproof lining package. The assembly is now ready to be incorporated in any of a number of footwear construction alternatives resulting in a waterproof product having an inherent comfort level due to the ability of the product to allow water vapor to exit the assembly while preventing water penetration from external sources while providing a dynamic fit, fluctuating with the wearer's variable weight load requirements.

The bottom 32 and the upper 34 are secured to each other in a conventional manner such as with the welt 72 shown in FIG. 4. However, the securing of the two portions needs to be done in a manner which does not nullify the structure and features of the waterproof breathable liner described above.

With the bottom 32 and the upper 34 secured together, with or without a waterproof, breathable liner, the chassis 36 is slipped into the shoe 30 to form part of the shoe 30.

As indicated above, the foot is dynamic in shape and the foot's shape changes as the foot is loaded and unloaded. The shoe having the bottom portion, the chassis, and the upper portion described allows for dynamic loading of the foot in comfort. The bottom portion expands and contracts with the expansion joints and the crowned shape. The chassis spreads the force on the foot over a greater area than the typical three loading points therefore reducing the pressure. The chassis likewise expands in shape because of the curve of the chassis in combination with the bottom. The upper portion expands and contracts as the foot moves. The combination of the structure and features of the bottom, the chassis, and the upper allows for a comfortable supportive shoe that also achieves a fashionable look.

While one embodiment has been described above with several alternative embodiments of the waterproof, breathable liner, it is recognized that alternative embodiments can achieve the same benefits. An alternative chassis 176 having four zones or regions is shown in FIG. 15A. In contrast to the chassis 36 shown in FIGS. 8A-10B, which has a single zone for both the heel and medial arch, the chassis 176 has a heel zone 178 and an arch zone 180. This is in addition to the chassis 36 having the metatarsal zone 182 and the toe zone 184. In addition, in contrast to the previous embodiment, a transition area 186 is located between the heel zone 178 and the arch zone 180 where the property characteristics change, typically from a firm resilient area in the heel zone 178 to a lower/soft durometer material. While the chart as represented in FIGS. 15B-15D shows transition through the entire zone 3, the arch zone, it is recognized that the transition can occur in a smaller area such as shown in FIG. 15A. The remainder of zone 3 can have uniform properties such as having a material with a medium hardness and an Asker C hardness rating of between 45-50 for the embodiment shown in FIG. 15C.

In one embodiment as schematically represented in FIG. 15B, the chassis 176 has a thickness of at least 6 millimeters. The heel zone 178 has a heel insert with an Asker C hardness rating of 45-50. Asker C is a scale similar to the durometer scale used to measure the hardness. The Asker C scale is typically used for soft material. (The use of the word insert in relation to FIGS. 15B-15D indicates that the component is added in a secondary operation, such as using an adhesive after the remaining layers are formed/molded together during the manufacturing process, such as a dual molding/extrusion process.) Overlying the insert is a second layer having a firm property with an Asker C hardness rating of 60-65. The arch zone 180 (zone 3) has a soft layer with an Asker C hardness rating of 30-35 that is overlaid by the firm layer. The total thickness stays constant, but the thickness of the firm upper layer tapers or thins out and the thickness of the soft layer increases as moving forward from the heel zone 178. The metatarsal zone 182 (zone 2) in this embodiment is formed of just the soft material. The final zone (zone 1), the toe zone 184 of the chassis 176 has a top layer of firm material and a bottom layer of soft material.

In an alternative embodiment as schematically represented in FIG. 15C, which could be considered a more comfortable and durable chassis 176, and typically more expensive chassis 176 to produce, the thickness of the chassis 176 is at least 8 millimeters. The heel zone 178 has a lower layer which is considered medium hardness and has an Asker C hardness rating of 45-50. The upper layer of the heel zone 178 is a firmer hardness material similar to the previous embodiment. The arch zone 180 has a medium hardness material which is overlied by the firm hardness material. The thickness of the firm hardness material tapers or thins out as in the previous embodiment. Both the metatarsal zone 182 and the toe zone 184 have a medium hardness material having an Asker C hardness rating of 45 to 50 as a lower layer. Inserts overlie or underlie the medium hardness material of the metatarsal zone 182 and the toe zone 184. The insert of the metatarsal zone 182 has a soft material having an Asker C hardness rating of 30-35, such as Poron™ or polyurethane. The insert of the toe zone 184 has a medium hardness material having an Asker C hardness rating of 45-50, such as Poron™ or polyurethane. While the materials of the inserts are similar, the material properties, such as hardness, are selected for the particular zone.

Still referring to FIG. 15C, the insert of the metatarsal zone 182 is a polyurethane that is characterized as a soft hardness material with an Asker C hardness rating of 30-35. The insert of the toe zone 184 is a polyurethane that is characterized as a medium hardness material with an Asker C hardness rating of 45-50. The reason that there are two layers in the toe zone that have the same material property is that one is added as an insert after the remaining layers are formed/molded together during the manufacturing process.

In an alternative embodiment as schematically represented in FIG. 15D, which could be considered a more comfortable and durable chassis 176 and typically a more expensive chassis 176 to produce than schematically shown in FIGS. 15B and 15C, the thickness of the chassis 176 is at least 10 millimeters. In contrast to the previous embodiments, the chassis 176 has a molded bottom 188 and an upper insert 190. The molded bottom 188 is made of a polyurethane having an Asker C hardness rating of 45-50. The upper insert 190 is a slow recovery material which means that the material conforms to the foot therein increasing the surface area of contact between the foot and the chassis 176 resulting in an improved pressure distribution. While conforming, the slow recovery material does recover slowly to its unloaded position, therefore the material partially recovers but still retains some or all of the shape of the foot as the foot goes through the gait cycle. The upper insert 190 in this embodiment is made of urethane such as slow recovery Poron™.

Interposed between the two layers, the molded bottom 188 and the upper insert 190, is a layer that has four zones, which defines the four zones of the chassis 176. The heel zone 178 has a material with a firm hardness. The arch zone 180, zone 3, has both material with a soft hardness and material with a firm hardness, similar to the first embodiment.

An alternative embodiment of a chassis 192 is shown in FIG. 15E. This embodiment is similar in some respects to that of the embodiment shown in FIGS. 8A-10B in that generally three zones are shown. The chassis 192 has a base or bottom layer 194, such as TPU, which extends the entire area. The chassis 192 is shown with an upper or top layer 196 of leather for the predominate purpose of style or fashion. Interposed is a layer 198 of varying materials or at least a material of varying properties.

In contrast to that of the embodiment shown in FIGS. 8A-10B, the chassis has areas of transition 200. For example, the heel and arch zones, zones 4 and 3, in FIG. 15E has a material with a medium hardness. The metatarsal zone has a material with a hardness rating characterized as soft. The transition from the “medium” material to the “soft” material occurs over a distance. Likewise, the transition from the metatarsal zone to the toe zone with the material having a “firm” characteristic occurs over a transition distance. In the heel and arch zone, a molded-in support insert can be included, such as shown in FIGS. 16A-18G and discussed below, to further tailor the properties.

A fifth alternative embodiment of chassis 202 having transition zones is shown in FIG. 15F. The chassis 202 has a single layer 204 of varying materials or at least a material of varying properties. In contrast to the previous embodiment, transition occurs over the entire heel zone 120 and the entire toe zone 124.

FIGS. 16A and 16B show a top and bottom view respectively of an alternative embodiment of a chassis 206 for a shoe 30. The chassis 206 has three zones or regions; a heel zone 208, a metatarsal zone 210, and a toe zone 212. The chassis 206 has an insert 214 that defines the heel zone 208.

In one embodiment, the insert 214 is made of polyurethane. The heel zone 208 in this embodiment includes the arch. The insert has a plurality of projections or fingers 216 that underlie the arch. The fingers 216 allow the insert 214 to flex more, therein creating a softer region than under the heel.

FIGS. 17A and 17B show sectional views of the chassis 206. The chassis 206 has a plurality of materials to form the chassis. As indicated above, the insert 214 on the medial side of the arch portion has a plurality of fingers 216. The fingers 216 create a series of slots 218.

Underlying the insert 214 and interposed between the fingers 216 in the slot 218 is a base material 220. Overlying the insert 214 is a top or finish material 222.

In addition, in contrast to the previous chassis, this chassis 206 has a cupped heel portion 226. The cupped heel portion 226 has an interior support layer 228 and a pair of outer coverings 230. As seen in FIG. 17A, the cupped heel portion 226 is fixed between the insert 214 and the top material 222.

With the cupped heel portion 226 being part of the chassis 206 in contrast to being formed in the upper 34, there is less likely to be rubbing between the foot 40 and the shoe 30 in the heel region. The chassis 206 moves with the foot 40 in contrast to the bottom 32 and the upper 34 whose movement is also influenced by the interaction with the ground.

A sectional view near the lateral side of the chassis 206 is shown in FIG. 17B. The insert 214 is interposed between the base material 220 and the top material 222. The cupped heel portion 226 is fixed between the insert 214 and the top material 222. While the base material 220 extends the entire length of the chassis 206, the upper layer transitions from the heel zone 208 to the metatarsal zone 210. The chassis 206 has a top material 234 in the metatarsal zone 210 which is distinct from the top material 222 in the heel zone 208.

A sectional view of the chassis 206 generally along the center line is shown in FIG. 18A. The three regions of the heel zone 208, the metatarsal zone 210, and the toe zone 212 are defined by the different materials used as a top material 222, 234, and 236.

As indicated above, the chassis 206 has three regions or zones, that of the heel zone 208, the metatarsal zone 210, and the toe zone 212. The top material 222 for the heel zone 208 is selected to be resilient. The top material 234 in the metatarsal zone 210 is selected to be a cushioning type material.

FIGS. 18B-18G show the cross sectional views at various locations of the chassis 206. In FIGS. 18F and 18G, the sides of the cupped heel portion 226 are seen. As indicated above, the cupped heel portion 226 is secured to the rest of the chassis 206 by being interposed between the insert 214 and the top material 222.

FIGS. 19A and 19B show the lateral side view and medial side view of the chassis 206 respectively. The cupped heel portion 226 projects upward from the rest of the chassis 206. The base material 220 is seen extending the length of the chassis 206. The top materials 222, 234, and 236 overlie the base material 220 in their respective zones.

A sectional view of a shoe 30 is shown in FIG. 19C. The shoe 30 has a bottom 32 and an upper 34. The upper 34 has an expansion portion 88. The upper 34 is sewn to the bottom 32 at the lip 116.

The shoe, in addition, has a chassis 206 similar to that shown in FIGS. 17A-19B. The chassis 206 has a plurality of zones including a heel zone 208, a metatarsal zone 210, and a toe zone 212. The chassis 206 has a cupped heel portion 226. In contrast to the previous embodiments, the cupped heel portion 226 has a tab 294 which extends through an opening 296 in the upper 34.

The interaction between the tab 294 and the upper 32 links or registers the chassis 206 with the upper 32. This registration prevents the chassis 206 from moving in the fore and aft direction relative to the upper 32 and the rest of the shoe 30. By registering the chassis 206 to the upper 32, the foot does not rub relative to the upper as the foot moves with the chassis 206. The registration of the chassis 206 with the upper 32 using the tab 294 allows the bottom 32 to expand as required and as discussed above and the chassis 206 to dynamically handle the pressure distribution to the foot.

It is recognized that registration of the chassis 206 to the rest of the shoe 30, the bottom 32 or upper 34, can be done by other techniques. For example, the chassis can have a rail or projection that is accepted in a complimentary void in the bottom. Furthermore, the mere fit of the chassis to the remainder of the shoe in certain embodiments results in registration.

FIGS. 19D and 19E show an alternative shoe that has a chassis 206 which includes a cupped heel portion 226. The upper 34 has a tab 338 that projects upward and has a fastener 340. The cupped heel portion 226 of the chassis 206 extends upward above the cuff 342 of the upper 34. The cuff 342 of the shoe in the heel has a concave cut as best seen in FIG. 19E. The cupped heel portion 226 has a fastener 344 which is complementary to the fastener 340 of the tab 298. The interaction between the fasteners 340 and 344 links or registers the chassis 206 with the upper 34.

An enlarged view of a linking mechanism 346 for registering the chassis 206 to the upper 34 is shown in FIG. 19F. The linking mechanism 346 of the chassis 206 overlies and secures to the upper 34 to register the chassis with the upper 34.

While the chassis is shown as detachable from the bottom, it is recognized that the distinctive zones, which are tailored for the respective part of the foot that interacts with the zone, can have the components of the chassis that are secured to the bottom 34. In the alternative, some of the components of the chassis can be secured to the bottom while other components of the chassis can be detachable and removable from the remainder of the shoe.

FIG. 20 shows an alternative shoe 240 having a bottom 242. The bottom 242 has a bottom surface or sole 244 that is not flat. The bottom surface 244 has a crown or concave surface wherein the edges, the lateral edge 246 and the medial edge 248 are raised compared to a center region 250. As the bottom 242 is loaded and the bottom surface 244 flattens, the lateral and medial edges 246 and 248 will move away from each other therein widening the shoe 240 in the region having this curvature. Typically the curvature is in the metatarsal zone and the toe zone.

Referring to FIG. 21A, the bottom surface 244 of the bottom 242 is shown. In contrast to the shoe shown in FIGS. 3 through 7B, the bottom 242 does not have an expansion portion to allow the edges to move apart.

FIG. 21B shows the top side of the bottom 242. The bottom 242 has an upper layer 256, in addition to an outer sole 254. The upper layer 256 has a heel and frame segment 258 and a pair of smaller inlays 260 and 262. The inlays 260 and 262 are adjacent to each other and are encircled by the frame segment portion of the heel and frame segment 258. One of the inlays 260 is in the toe zone and the other inlay 262 is in the metatarsal zone. In the bottom 242, there is a supporting shank 264 shown in hidden line which extends from the heel region through the arch zone and in proximity to the metatarsal zone. In this embodiment, the inlays 260 and 262 form zones in the bottom 242 having a purpose similar to those of the zones in the chassis as discussed above.

A sectional view taken generally along the center line of the bottom 242 is shown in FIG. 22A. The bottom 242 has the outer sole 254 with the bottom surface 244. The heel and frame segment 258 of the upper layer 256 is shown in the heel and arch region and at the front of the shoe. Within the heel and frame segment 258 is the shank 264 which stiffens the arch zone of the shoe 30. The metatarsal zone and toe zone have respectively different inlays 260 and 262.

In a preferred embodiment, the outer sole 254 is made of rubber for its wear and slip resistance properties. The inlay 262 in the toe zone is made of a polyurethane having an Asker C hardness of 45-50. The inlay 260 in the metatarsal zone is made of a polyurethane having a hardness of 30 Asker C. Both inlays, 260 and 262 are selected for cushioning and resilience properties. The supporting shank 264 in a preferred embodiment is made of a molded or stamped stiff material such as nylon, TPU, or steel.

FIGS. 22B-22E show sectional views of the bottom 242. The bottom 242 has a lip 266 for securing to the upper 34. The lip 266 is carried by the heel and frame segment 258 of the upper layer 256.

As seen in FIG. 22B, the bottom surface 244 of the outer sole 254 is not flat and is crowned. The edges 246 and 248 of the bottom surface 244 of the bottom 242 are raised compared to the central region 250. When a load, such as stepping is applied to the bottom 242 of the shoe 30, the bottom surface 244 flattens therein moving the lateral and medial edges 246 and 248 apart. As indicated above, one of the benefits of the extension of the metatarsal zone of the shoe besides comfort is that the shoe 30 has a more tailored look in the unloaded position.

FIGS. 22C, 22D, and 22E each show cross sections of the bottom 242. The supporting shank 264 extends through the heel and frame segment 258 of the upper layer 256. While the bottom surface 244 has a slight curvature in the heel zone as seen in FIGS. 22D and 22E, the slight curvature in combination with the increased thickness of the upper layer 256 results in the heel zone expanding only slightly as compared to the metatarsal zone.

In contrast to the embodiment shown in FIGS. 3-7B, the upper is not sewn with a horizontal stitch through the welt to the side. In contrast as seen in FIG. 22B1, the upper is sewn through to the sole 254 of the bottom 242 with a line of stitching 268. The lip 266 in this embodiment helps retain the shape of the upper 34. A chassis 36 is shown overlying the bottom 242.

An enlarged view of the lip 266 is seen in FIG. 22D1. The upper 34 is sewn with a vertical stitch with the upper 34 located inboard of the lip 266.

Referring to FIGS. 23A and 23B, the medial and lateral side of the bottom 242 is shown. Both the outer sole 254 and the heel and frame segment 258 of the upper layer 256 are seen. The lip 266 which helps retain the upper 34 is likewise shown.

Referring to FIG. 24B, an alternative bottom 272 has an inlay 274 which is encircled by a heel and frame segment 278 of an upper layer 276. However in contrast to the previous embodiment, the inlay 274 is a displacement material, which while located above the outer sole 280, extends through ports 282, as seen in FIG. 25C, toward a bottom surface 284 of the outer sole 280. As the foot 40 applies pressure to the chassis 36, not shown in this figure, the inlay 274, the displacement material, is forced into soling channels 286 of the shoe 30 as seen in FIG. 24A therein creating more space within the shoe.

FIG. 25A is a sectional view taken generally along the center line of the bottom 272 of the shoe 30 showing that the outer sole 280 of the bottom 272 has the bottom surface 284 similar to the previous embodiment. In addition, the heel and frame segment 278 of the upper layer 276 receives a supporting shank 264. The inlay 274 or displacement material is shown extending through the ports or apertures 282 to the soling channels or grooves 286 on the bottom surface 284 of the outer sole 280.

FIGS. 25B and 25C are sectional views of the metatarsal zone showing the displacement material, inlay 274, overlying the outer sole 280 and extending through the apertures 282 to the grooves 286 in the bottom surface 284.

Referring to FIG. 26, a shoe 350 with an alternative embodiment of an expanding bottom 352 is shown. The shoe 350 has an upper 34, a bottom 352, and a chassis 36. The bottom 352 has a base or sole portion 354 having a void 356 in which an inner layer 358 is located. Interposed between the inner layer 358 of the bottom 352 and the base 354 of the bottom 352, a cavity 360 is formed. The cavity 360 is bounded on top by the inner layer 358 therein forming an upper surface 362. The upper surface 362 has a planar outer edge 364 and a convex center portion 366. Located within a center portion 368 of the cavity 360 underlying the convex center portion 366 of the upper surface 362 is an inlay or displacement material 370 similar to that described above with respect to FIGS. 24A-25C. This inlay or displacement material 370 expands into exterior portions 372, underlying the planar outer edge 364 of the upper surface 362 of the cavity 360, when force is applied to the inner layer 358 of the bottom 352. This force is applied through the chassis as the person loads the shoe. As the displacement material 368 expands into the exterior portion 372, the inner layer 358 and likewise the chassis 36 can move downward in the shoe 350, therein creating more space within the shoe. Likewise, as the force is removed, the inlay or displacement material contracts back into the center chamber forcing the inner layer upward therein reducing or decreasing the volume within the shoe.

In manufacturing this shoe, the upper 34 is secured by a line of stitching 376 to the inner layer 358 prior to the base or outer sole 354 of the bottom 352 being molded such as by direct injection or vulcanized to the inner layer 358 of the bottom 352 and the upper 34. In one embodiment, the inlay material 370 is a polymergel having exponential elongation properties with memory, that is molded or extruded into a base shape. The midsole is premolded into a convex shape and allows for shape displacement. The cavity 360 with the inlay 370 is typically located in the metatarsal zone and toe zone of the shoe 350.

FIG. 27A is a front sectional view of a shoe 380 with another alternative bottom construction. The shoe 380 has an upper 34, a bottom 382 and a chassis 36. The bottom 382 has a base layer 384 and an upper layer 386. The upper layer 386 has a rigid outer portion 388 which underlies the chassis 36 in the shoe 380 except for a void 390 in the metatarsal zone. This central void 390 in the metatarsal zone receives a rigid inlay 392. In a preferred embodiment, the rigid inlay 392 is a polyurethane or a TPU. The inlay 392 is secured to the outer portion 388 by a plurality of elastic membranes 394 which allow the outer layer 388 to expand away from the inlay 392 as the foot expands. By the outer portion 388 and the inlay 392 being of a rigid material in the upper layer 386, an external concentrated load or pressure point such as from a rock is dissipated over a larger area by this rigid inner layer therein reducing the pressure to the foot. Expansion or movement can be provided by a crown and mechanical grooves.

FIG. 27B is a top view of the bottom 382 of the shoe 380 showing the connections by the elastic membranes 394 of the inlay 392 to the rigid outer layer 388. This dissipation of pressure by spreading the force over an area results in ground insulation, i.e., the ground surface is not perceived.

It is recognized that while the expansion of the bottom of the shoe has been shown in the embodiments to expand in the lateral direction, the shoe can be designed to expand lengthwise also. Lengthwise expansion may be desirable in children's shoes so that the shoe has some room for growth. One method of expansion is to have slots in a first material which receive a second material, such as in the first embodiment, extending in a lateral direction.

As indicated above the foot is dynamic in shape and the foot's shape changes as the foot is loaded and unloaded. The shoe having the bottom portion, the chassis, and the upper portion as described allows the dynamic movement of the foot in comfort. The bottom portion expands and contracts with the expansion joints and the crowned shape. The chassis spreads the force on the foot over a greater area than the typical three loading points therefore reducing the pressure and likewise expanding in shape because of the curve of the chassis in combination with the bottom. The upper portion expands and contracts as the foot moves. The combination of the structure and features of the bottom portion, the chassis, and the upper portion allows for a comfortable supporting shoe that also achieves a fashionable look.

While this invention has been particularly shown and described with references to preferred embodiments thereof, it will be understood by those skilled in the art that various changes in form and details may be made therein without departing from the scope of the invention encompassed by the appended claims. For example, various features of different embodiments can be combined or omitted. In some embodiments, more than one expansion mechanism is provided, whereby each expansion mechanism separately contributes to the expansion of the shoe. Additionally, some embodiments of the present invention shoe can have a lower or bottom portion that includes a bottom and a chassis, while other embodiments can have a lower or bottom portion that has a bottom but no chassis.

1. Footwear, comprising
a bottom;
an upper carried by the bottom to define a foot receiving cavity; and
a chassis inserted in the foot receiving cavity in overlying relationship to the bottom;
the bottom having an upper layer and a lower layer, the upper layer having an opening defining a void space between the lower layer and the chassis, and at least one insert assembled in the void space, the upper layer being formed from a first material and the insert being formed from a second material different from the first material.

2. The footwear as claimed in claim 1, wherein the opening in the upper layer is in a position underlying the forefoot of a foot when the foot is in the foot receiving cavity.

3. The footwear as claimed in claim 2, wherein the insert includes a first inlay and a second inlay, the first inlay being formed from one material and the second inlay being formed from another material different from the one material.

4. The footwear as claimed in claim 1, wherein the insert includes a first inlay and a second inlay, the first inlay being formed from one material and the second inlay being formed from another material different from the one material.

5. The footwear as claimed in claim 4, wherein the first inlay is in a position underlying the toes of a foot when the foot is in the foot receiving cavity, and the second inlay is in a position underlying the metatarsals of the foot when the foot is in the foot receiving cavity.

6. The footwear as claimed in claim 5, wherein the one material has a first degree of hardness and the another material has a degree of hardness less than the first degree of hardness.

7. The footwear as claimed in claim 4, wherein the one material is a first polyurethane and the another material is a polyurethane different from the first polyurethane.

8. The footwear as claimed in claim 1, wherein the lower layer of the bottom is formed from rubber.

9. The footwear as claimed in claim 1, wherein the insert is formed from a displacement material and the lower layer includes a bottom surface and a plurality of through ports extending from the upper layer to the bottom surface, the displacement material having a degree of deformability such that a portion of the displacement material is displaced into at least some of the through ports as a load is applied to the displacement material in a direction toward the lower layer.

10. Footwear, comprising
a bottom;
an upper carried by the bottom to define a foot receiving cavity; and
a chassis inserted in the foot receiving cavity in overlying relationship to the bottom;
the bottom having an upper layer, a lower layer, a void space defined between the upper layer and the lower layer, and a displacement material positioned in the void space.

11. The footwear as claimed in claim 10, wherein the void space is in a position underlying the forefoot of a foot when the foot is in the foot receiving cavity.

12. The footwear as claimed in claim 10, wherein, when no downward load is applied to the upper layer of the bottom, the void space has an initial volume and the displacement material has a volume less than the initial volume.

13. The footwear as claimed in claim 10, wherein a bottom surface of the upper layer includes a generally concave recess, and the displacement material is positioned below the generally concave recess.
